(19) United States
(12) Patent Application Publication
(10) Pub. No.: US 2026/0076563 A1
(43) Pub. Date: Mar. 19, 2026

(54) SYSTEMS AND METHODS FOR IDENTIFYING VISUAL STRESSORS IN OFFICE ENVIRONMENTS AND RECOMMENDING ERGONOMIC ADJUSTMENTS

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/885,399

(22) Filed: Sep. 13, 2024

(51) Int. Cl. A61B 3/113; A61B 3/00

(52) U.S. Cl. CPC A61B 3/113; A61B 3/005

(57) ABSTRACT

A user's visual health can be improved via a virtual reality (VR) system, which can include a VR headset in electronic communication with a computing device. The computing device causes virtual environments, which can include features such as furniture, lighting, and equipment, to be displayed on the VR headset. Using varying combinations of eye-tracking sensors, eye-tracking cameras, motion-tracking sensors, handheld devices, and microphones, the VR headset collects data about the user as she completes visual tasks in the virtual environment as the features are changed. Optionally, the virtual environment is the user's workplace, and she completes various tasks in the virtual workplace to determine an ergonomically ideal workplace arrangement. Optionally, advanced algorithms in the computing device can dynamically alter the features to optimize the user's visual and physical comfort in her workplace.

### BACKGROUND

### Field of the Inventions

The present application relates to methods of assessing various ocular conditions through extended reality systems. More specifically, methods and systems are applied to conduct visual tasks and exams in extended reality environments to evaluate patients for ocular conditions and diseases, such as eye misalignment and visual processing disorders.

### Description of the Related Art

As virtual reality (VR) technology has become increasingly sophisticated, new highly immersive experiences have been made possible through improvements in head and motion tracking systems. Eye-tracking technology allows systems to detect and respond to where the user is looking. This capability enhances user interaction and makes virtual environments more responsive and engaging. Eye tracking is being integrated into a variety of VR applications, from gaming and training simulations to medical diagnostics and research, as it offers a more intuitive way for users to interact with digital content.

### SUMMARY

The systems, methods and devices of this disclosure each have several innovative aspects, no single one of which is solely responsible for the desirable attributes disclosed herein.

Despite the advancements in VR technology, and in particular, eye-tracking technology, in accordance with some embodiments disclosed herein is the realization that VR technology can provide unique eyecare solutions through monitoring and tracking one or more of the user's eyes and providing a diagnostic, treatment protocol, and/or treatment system. Indeed, in accordance with some embodiments disclosed herein is the realization that VR technology can be used to address challenges associated with diagnosing a variety of eye disorders and ocular conditions, such as detecting misalignment, macular degeneration, tear film characteristics, floater characteristics, eye tracking issues, motion sensitivity, and other eye movement disorders and the treatment of such.

In some embodiments, a virtual reality (VR) assessment of rapid near-far focusing ability can be conducted through engaging, gamified tasks. This method can be implemented through a system that can comprise a high-resolution VR headset integrated with precision eye-tracking technology and specialized software capable of generating dynamic virtual environments. Users may wear the VR headset and participate in a series of interactive games that require them to frequently and quickly shift their focus between near and far objects. The eye-tracking sensors monitor the user's eye movements, focus adjustments, and reaction times, while the software analyzes these responses to provide a comprehensive assessment of the user's near-far focusing ability.

Optionally, a VR software can employ a variety of gamified tasks, such as catching objects that move rapidly from near to far distances, shooting targets that appear at varying depths, and navigating through obstacle courses that require quick adjustments in focus. These tasks are designed to be engaging and entertaining to ensure that users remain motivated throughout the assessment. The software processes the data in real-time using advanced algorithms to evaluate parameters such as focusing speed, accuracy, and consistency. The results are compiled into a detailed report that highlights the user's focusing performance, identifying any deficiencies that could indicate conditions such as accommodative dysfunction or presbyopia. This method offers a dynamic, engaging, and precise approach to assessing rapid near-far focusing ability in a controlled virtual environment.

The VR-based focusing ability assessment system can utilize a high-quality VR headset, such as the Oculus Quest 2, which may be integrated with high-precision eye-tracking technology. Eye-tracking sensors can include infrared cameras capable of capturing detailed eye movements and focus adjustments with high accuracy and minimal latency. The system can implement a software program that assists in creating a library of gamified tasks designed to test different aspects of near-far focusing ability. These tasks can include scenarios where users must rapidly shift their focus to catch moving objects, shoot targets at varying depths, and navigate through virtual environments that challenge their ability to adjust focus quickly and accurately.

The system can be calibrated using a control group of individuals with known focusing abilities to establish baseline performance metrics and validate the accuracy of the assessment algorithms. Users can then operate the system by wearing the VR headset and participating in the guided gamified tasks within the virtual environments. The eye-tracking sensors monitor their eye movements and focus adjustments, while the software records and analyzes the data in real-time. The user can receive a detailed report outlining their focusing performance, highlighting any deviations from normal patterns, and providing recommendations for further optometric consultation if necessary. This approach offers a precise, non-invasive, and user-friendly method for assessing rapid near-far focusing ability, representing a significant advancement over traditional testing techniques and providing substantial benefits for both clinical and research applications.

In some embodiments, a VR method for training and strengthening ocular muscles can be performed using targeted exercises. This system can comprise a high-resolution VR headset integrated with precision eye-tracking technology and specialized software capable of generating interactive exercises that specifically target the ocular muscles. Users may wear the VR headset and engage in a series of visually stimulating tasks that require precise and controlled eye movements, such as tracking moving objects, focusing on targets at varying distances, and performing coordinated eye movement patterns. The eye-tracking sensors continuously monitor the user's eye movements, providing real-time feedback and adjusting the difficulty level of the exercises to ensure optimal training intensity.

Optionally, the VR system can include a software comprising a variety of exercises designed to improve different aspects of ocular muscle strength and coordination, such as saccades (rapid eye movements between points), pursuits (smooth tracking of moving objects), and vergence (simultaneous inward or outward movement of both eyes). These exercises are embedded in engaging and gamified scenarios to maintain user motivation and interest. The software processes the data in real-time using advanced algorithms to track progress and adapt the exercises to the user's performance. The results are compiled into a comprehensive report that provides insights into the user's ocular muscle strength and coordination, highlighting areas of improvement and offering recommendations for continued training. This method offers a dynamic, engaging, and precise approach to ocular muscle training, representing a significant advancement over traditional eye exercise techniques.

The VR-based ocular muscle training system can utilize a high-quality VR headset, which may be integrated with high-precision eye-tracking technology. The eye-tracking sensors can include infrared cameras capable of capturing detailed eye movements with high accuracy and minimal latency. The system can implement a software program that assists in creating a library of targeted exercises designed to train different ocular muscles. These exercises can include scenarios where users must follow moving objects, shift focus between near and far targets, and perform coordinated eye movements within a virtual environment.

The system can be calibrated using a control group of individuals with varying levels of ocular muscle strength to establish baseline performance metrics and validate the effectiveness of the training algorithms. Users can then operate the system by wearing the VR headset and participating in the guided exercises within the virtual environments. The eye-tracking sensors monitor their eye movements and provide real-time feedback, while the software records and analyzes the data to track progress and adapt the difficulty of the exercises. The user can receive a detailed report outlining their ocular muscle performance, highlighting improvements and areas needing further attention, and providing recommendations for continued training. This approach offers a precise, non-invasive, and user-friendly method for strengthening ocular muscles, providing substantial benefits for both clinical therapy and personal eye care routines.

In some embodiments, a VR method designed for vision test training can be performed by using feedback-adjusted visual challenges. This system can comprise a high-resolution VR headset integrated with precision eye-tracking technology and specialized software capable of generating interactive visual tests that adapt based on real-time feedback. Users may wear the VR headset and engage in a series of vision training exercises, such as reading charts, identifying objects at varying distances, and tracking moving stimuli. The eye-tracking sensors monitor the user's eye movements, focus adjustments, and response times, while the software dynamically adjusts the difficulty level of the visual challenges based on the user's performance. This adaptive approach ensures that the training remains challenging and effective, tailored to the individual's visual capabilities.

Optionally, the VR software can include a variety of vision tests designed to improve different aspects of visual acuity, depth perception, and eye coordination. These tests are presented in engaging and interactive formats to maintain user motivation and interest. For instance, users might read from a virtual Snellen chart, identify and select objects that appear at random locations, or follow a moving target through a complex path. The software processes the data in real-time, using advanced algorithms to analyze performance metrics such as accuracy, speed, and stability of eye movements. The results are compiled into a comprehensive report that provides insights into the user's vision training progress, highlighting improvements and areas needing further attention. This method offers a dynamic, engaging, and precise approach to vision test training, representing a significant advancement over traditional, static vision exercises.

The VR-based vision test training system may utilize a high-quality VR headset, which may be integrated with high-precision eye-tracking technology. The eye-tracking sensors can include infrared cameras capable of capturing detailed eye movements, fixation points, and response times with high accuracy and minimal latency. The system can implement a software program that assists in creating a library of visual tests and training exercises designed to improve various aspects of vision. These exercises can include scenarios where users must read from virtual charts, identify objects at different distances, and track moving stimuli within a virtual environment. Each task is designed to adapt in real-time based on user performance, ensuring an individualized training experience.

The system can be calibrated using a control group of individuals with diverse visual profiles to establish baseline performance metrics and validate the effectiveness of the adaptive training algorithms. Users can then operate the system by wearing the VR headset and participating in the guided vision tests and training exercises within the virtual environments. The eye-tracking sensors monitor their eye movements and responses to the visual stimuli, while the software records and analyzes the data to adjust the difficulty of the tasks in real-time. The user can receive a detailed report outlining their vision training performance, highlighting improvements and areas needing further attention, and providing recommendations for continued training. This approach offers a precise, non-invasive, and user-friendly method for vision test training, providing substantial benefits for both clinical therapy and personal vision improvement routines.

In some embodiments, a VR system can assess the impact of environmental factors on vision using simulated exposures. This system can comprise a high-resolution VR headset integrated with precision eye-tracking technology and specialized software capable of generating realistic virtual environments that simulate various environmental conditions. Users may wear the VR headset and engage in a series of tasks that involve visual assessments under different simulated environmental factors, such as varying light levels, glare, fog, smoke, and wind. The eye-tracking sensors monitor the user's gaze direction, fixation duration, and visual acuity, while the software analyzes these responses to provide a comprehensive assessment of how different environmental factors affect visual performance.

Optionally, the VR software can include a variety of scenarios where users experience and respond to different environmental conditions. For example, users might perform visual tasks under low-light conditions, navigate through foggy environments, or identify objects while exposed to simulated glare from bright lights. These tasks are designed to challenge the user's vision and simulate real-world conditions that can affect visual performance. The software processes the data in real-time using advanced algorithms to evaluate parameters such as reaction time, accuracy, and stability of eye movements under each condition. The results are compiled into a comprehensive report that provides insights into the user's visual performance across different environmental exposures, identifying any deficiencies that could indicate vulnerabilities to specific conditions. This method offers a dynamic, engaging, and precise approach to assessing the impact of environmental factors on vision, representing a significant advancement over traditional vision testing techniques.

The VR-based environmental vision assessment system may utilize a high-quality VR headset, which may be integrated with high-precision eye-tracking technology. The eye-tracking sensors can include infrared cameras capable of capturing detailed eye movements and fixation patterns with high accuracy and minimal latency. The system can implement a software program that assists in creating a library of virtual environments designed to simulate various environmental conditions. These environments can include scenarios where users must perform visual tasks under different light levels, glare, fog, smoke, and wind, among other factors.

The system can be calibrated using a control group of individuals with known visual profiles to establish baseline performance metrics and validate the accuracy of the environmental simulation algorithms. Users can then operate the system by wearing the VR headset and participating in the guided visual tasks within the simulated environments. The eye-tracking sensors monitor their eye movements and responses to the environmental stimuli, while the software records and analyzes the data in real-time. The user can receive a detailed report outlining their visual performance under each environmental condition, highlighting any deviations from normal patterns, and providing recommendations for further optometric consultation if necessary. This approach offers a precise, non-invasive, and user-friendly method for assessing the impact of environmental factors on vision, providing substantial benefits for both clinical and research applications.

In some embodiments, a VR method evaluates the effectiveness of eye exercises by comparing a user's pre- and post-training vision. This system can comprise a high-resolution VR headset integrated with precision eye-tracking technology and specialized software capable of conducting comprehensive vision assessments before and after a series of targeted eye exercises. Users may wear the VR headset and participate in initial vision assessments that can include tasks such as reading charts, identifying objects at various distances, and tracking moving stimuli. The eye-tracking sensors monitor the user's eye movements, focus adjustments, and visual acuity, while the software records these baseline measurements.

Optionally, following the initial assessment, users engage in a prescribed regimen of eye exercises within the virtual environment, designed to improve various aspects of visual function such as convergence, accommodation, and saccadic movements. These exercises are interactive and gamified to maintain user engagement and ensure consistent training. After completing the exercise regimen, users undergo a second round of vision assessments identical to the initial tests. The software then compares the pre- and post-training data using advanced algorithms to evaluate improvements in visual performance. The results are compiled into a detailed report that highlights changes in visual acuity, eye coordination, and focusing ability, providing clear insights into the effectiveness of the eye exercises. This method offers a dynamic, engaging, and precise approach to assessing the impact of eye exercises on visual function, representing a significant advancement over traditional evaluation techniques.

The VR-based vision evaluation system may utilize a high-quality VR headset, which may be integrated with high-precision eye-tracking technology. The eye-tracking sensors can include infrared cameras capable of capturing detailed eye movements, fixation points, and focus adjustments with high accuracy and minimal latency. The system can implement a software program that assists in creating a comprehensive library of vision assessments and eye exercises designed to improve different aspects of visual function. The initial vision assessments can include scenarios where users must read virtual charts, identify objects at various distances, and follow moving targets within a virtual environment.

Prior to use, the system can calibrate the hardware and software components using a control group of individuals with varying visual profiles to establish baseline performance metrics and validate the accuracy of the assessment and training algorithms. Users can then operate the system by wearing the VR headset and participating in the guided initial vision assessments. After completing the prescribed regimen of interactive eye exercises, users undergo a second round of vision assessments. The eye-tracking sensors monitor their eye movements and responses to the visual tasks, while the software records and analyzes the pre- and post-training data in real-time. The user can receive a detailed report outlining the effectiveness of the eye exercises, highlighting improvements and areas needing further attention, and providing recommendations for continued training or optometric consultation if necessary. This approach offers a precise, non-invasive, and user-friendly method for evaluating the effectiveness of eye exercises, providing substantial benefits for both clinical therapy and personal eye care routines.

Some embodiments in the present application are directed to a VR method for identifying visual stressors in office environments and recommending ergonomic adjustments. This system can comprise a high-resolution VR headset integrated with precision eye-tracking technology and specialized software capable of simulating realistic office settings. Users may wear the VR headset and navigate through a virtual office environment that replicates common workplace conditions, such as screen glare, poor lighting, and suboptimal workstation setups. The eye-tracking sensors monitor the user's gaze direction, fixation duration, and blink rate, while the software analyzes these responses to identify sources of visual stress and discomfort.

Optionally, the system can include software equipped with a variety of scenarios that simulate different office layouts, lighting conditions, and screen configurations. Users perform typical office tasks, such as reading documents, working on spreadsheets, and attending virtual meetings. The software processes the data in real-time, using advanced algorithms to evaluate parameters such as visual fatigue, eye strain, and ergonomic posture. Based on the analysis, the system provides personalized recommendations for ergonomic adjustments, such as optimizing screen height and angle, improving lighting conditions, and rearranging workstation elements to reduce visual stress. The results are compiled into a comprehensive report that offers actionable insights to enhance visual comfort and productivity in the office environment. This method offers a dynamic, engaging, and precise approach to identifying and mitigating visual stressors in workplace settings, representing a significant advancement over traditional ergonomic assessments.

The VR-based visual stressor identification and ergonomic adjustment system can utilize a high-quality VR headset, which may be integrated with high-precision eye-tracking technology. The eye-tracking sensors can include infrared cameras capable of capturing detailed eye movements, fixation patterns, and blink rates with high accuracy and minimal latency. The system can implement a software program that assists in creating a library of virtual office environments that simulate various workplace conditions, including different lighting setups, screen positions, and office layouts.

The system can be calibrated using a control group of individuals with diverse visual and ergonomic profiles to establish baseline performance metrics and validate the accuracy of the stressor identification algorithms. Users can then operate the system by wearing the VR headset and performing guided office tasks within the virtual environments. The eye-tracking sensors monitor their eye movements and responses to the simulated office conditions, while the software records and analyzes the data in real-time. The user can receive a detailed report outlining the identified visual stressors and providing personalized ergonomic recommendations to enhance visual comfort and reduce eye strain. This approach offers a precise, non-invasive, and user-friendly method for identifying visual stressors and recommending ergonomic adjustments in office environments, providing substantial benefits for both workplace health and productivity.

In some embodiments, a VR method is used for evaluating a user's optical response to ultraviolet (UV) exposure and recommending appropriate protective measures. This system can comprise a high-resolution VR headset integrated with precision eye-tracking technology and specialized software capable of simulating various levels of UV exposure within immersive virtual environments. Users may wear the VR headset and engage in a series of tasks and scenarios that simulate exposure to UV light under different conditions, such as sunny outdoor settings, reflections from water surfaces, and high-altitude environments. The eye-tracking sensors monitor the user's gaze direction, blink rate, and pupillary responses, while the software analyzes these responses to assess the impact of UV exposure on the eyes.

Optionally, the VR software can include a variety of scenarios designed to mimic real-world UV exposure situations. For instance, users might perform tasks that require prolonged outdoor activity, interaction with reflective surfaces, or movement through shaded and unshaded areas. The software processes the data in real-time, using advanced algorithms to evaluate parameters such as blink frequency, squinting, and pupil constriction. Based on the analysis, the system provides personalized recommendations for protective measures, such as the optimal type of sunglasses, UV-blocking contact lenses, or specific behavioral adjustments to minimize UV exposure. The results are compiled into a comprehensive report that offers actionable insights for protecting the eyes from UV damage. This method offers a dynamic, engaging, and precise approach to evaluating and mitigating the effects of UV exposure on ocular health, representing a significant advancement over traditional assessment techniques.

The VR-based UV exposure assessment tool can utilize a high-quality VR headset, which may be integrated with high-precision eye-tracking technology. The eye-tracking sensors can include infrared cameras capable of capturing detailed eye movements, blink rates, and pupillary responses with high accuracy and minimal latency. The system can implement a software program that assists in creating a library of virtual scenarios that simulate various UV exposure conditions, including outdoor activities, reflective surfaces, and varying light intensities.

The system can be calibrated using a control group of individuals with known ocular health profiles to establish baseline performance metrics and validate the accuracy of the UV exposure assessment algorithms. Users can then operate the system by wearing the VR headset and participating in the guided UV exposure tasks within the virtual environments. The eye-tracking sensors monitor their eye movements and responses to the simulated UV light conditions, while the software records and analyzes the data in real-time. The user can receive a detailed report outlining the eye's response to UV exposure and providing personalized recommendations for protective measures to mitigate UV damage. This approach offers a precise, non-invasive, and user-friendly method for assessing and protecting against UV exposure, providing substantial benefits for both clinical applications and personal eye care routines.

The disclosures of the following applications are incorporated by reference herein, in their entirety: U.S. application Ser. No. 18/811,673, filed on Aug. 21, 2024; U.S. application Ser. No. 18/811,677, filed on Aug. 21, 2024; U.S. application Ser. No. 18/811,683, filed on Aug. 21, 2024; U.S. application Ser. No. 18/811,686, filed on Aug. 21, 2024; U.S. application Ser. No. 18/811,690, filed on Aug. 21, 2024; U.S. application Ser. No. 18/811,694, filed on Aug. 21, 2024; U.S. application Ser. No. 18/811,695, filed on Aug. 21, 2024; U.S. application Ser. No. 18/811,698, filed on Aug. 21, 2024; U.S. application Ser. No. 18/811,701, filed on Aug. 21, 2024; U.S. application Ser. No. 18/811,704, filed on Aug. 21, 2024; U.S. application Ser. No. 18/811,713, filed on Aug. 21, 2024; U.S. application Ser. No. 18/811,715, filed on Aug. 21, 2024; U.S. application Ser. No. 18/811,720, filed on Aug. 21, 2024; U.S. application Ser. No. 18/811,724, filed on Aug. 21, 2024; U.S. application Ser. No. 18/811,729, filed on Aug. 21, 2024; and U.S. application Ser. No. 18/811,730, filed on Aug. 21, 2024.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is an example data processing environment having one or more servers communicatively coupled to one or more computer devices, in accordance with some embodiments.

FIG. 2 is an environment in which a computer device is applied to facilitate visual assessment or eyewear fitting, in accordance with some embodiments.

FIG. 3 is a block diagram of a computer system configured to implement vision assessment or eyewear fitting, in accordance with some embodiments.

FIG. 4 is a block diagram of a machine learning system for training and applying machine learning models, in accordance with some embodiments.

FIG. 5A is a structural diagram of an example neural network applied to process input data in a machine learning model, in accordance with some embodiments.

FIG. 5B is an example node in the neural network, in accordance with some embodiments.

FIG. 6A is an example “tumbling E” chart applied in a visual acuity test, in accordance with some embodiments.

FIGS. 6B, 6C, 6D, and 6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D can include four diagrams of example graphical user interfaces rendered to determine a visual acuity score in a virtual environment created by a headset device, in accordance with some embodiments.

FIGS. 9A-9C can include three diagrams of example graphical user interfaces rendered to determine a nearsighted or farsighted power in a virtual environment created by a headset device, in accordance with some embodiments.

FIGS. 10A-10F can include six diagrams of example graphical user interfaces rendered to determine eye stigmatism in a virtual environment created by a headset device, in accordance with some embodiments.

FIG. 11 illustrates a normal pupil, a constricted pupil, and a dilated pupil, as perceived by a VR headset, in accordance with some embodiments.

FIG. 12 illustrates charts of the cardinal gaze positions labeled with the extraocular muscles that correspond with the gaze positions superimposed over the patient's eyes, in accordance with some embodiments.

FIG. 13A illustrates a virtual environment that can include an object that can be positioned at different distances from the patient, in accordance with some embodiments.

FIG. 13B illustrates a virtual environment that can include multiple objects positioned at different distances from the patient, in accordance with some embodiments.

FIG. 14A illustrates an array of optotypes with different contrasts relative to the virtual environment, colors, orientations, and sizes, in accordance with some embodiments.

FIGS. 14B-1 and 14B-2 illustrate a patient reading a vision chart at two different distances, in accordance with some embodiments.

FIG. 15A illustrates a virtual environment that is partially obscured by glare, in accordance with some embodiments.

FIG. 15B illustrates a virtual environment that is partially obscured by fog and smoke, in accordance with some embodiments.

FIG. 16 illustrates a placement of motion-tracking sensors on a VR headset, in accordance with some embodiments.

FIG. 17 illustrates a virtual office space that can be furnished and arranged in VR for optimized productivity and comfort, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description can include specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

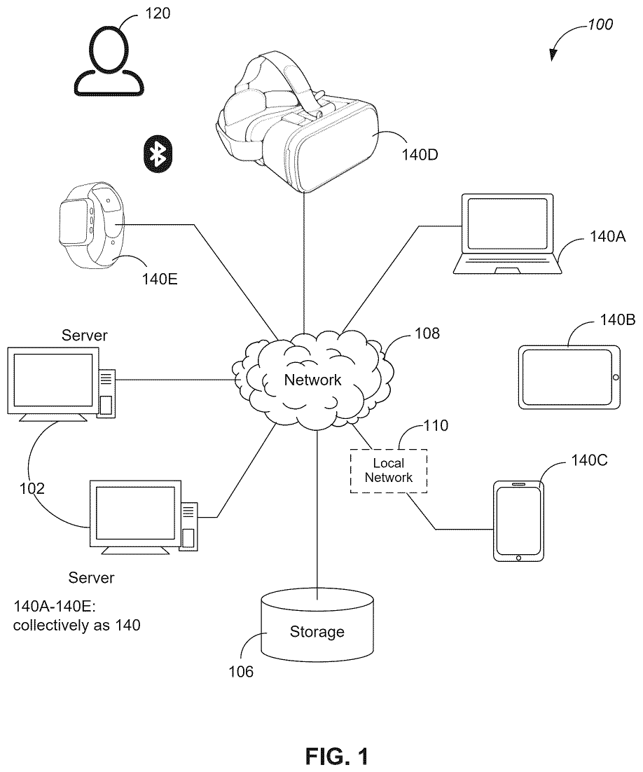

Referring now to the figures, FIG. 1 is an example data processing environment 100 having one or more servers 102 communicatively coupled to one or more computer devices 140 (e.g., a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera).

In some implementations, the one or more computer devices 140 can include a headset device 140D (also called a head-mounted display (HMD) device 140D) configured to render extended reality content. In some implementations, the one or more computer devices 140 can include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 can provide system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 can further include a storage 106 for storing data related to the servers 102, computer devices 140, and applications executed on the computer devices 140. For example, storage 106 may store video content, static visual content, and/or audio data.

The one or more servers 102 can enable real-time data communication with the computer devices 140 that can be remote from each other or from the one or more servers 102. Further, in some embodiments, the one or more servers 102 can implement data processing tasks that are not completed locally by the computer devices 140.

For example, the computer devices 140 can include a game console (e.g., the headset device 140D) that executes an interactive online gaming application (e.g., for visual assessment or eyewear fitting). The game console can receive a user instruction and sends it to a server 102 with user data. The server 102 generates a stream of video data based on the user instruction and user data and provides the stream of video data for display on the game console and other computer devices that can be engaged in the same session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 can include connections, such as wire, wireless communication links, or fiber optic cables. Examples of the one or more communication networks 108 can include local area networks (LAN), wide area networks (WAN) such as the Internet, or a combination thereof. The one or more communication networks 108 are, optionally, implemented using any known network protocol can include various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 108 may be established either directly (e.g., using 1G/4G connectivity to a wireless carrier), or through a network interface 110 (e.g., using a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D can include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the headset device 140D. In some embodiments, the microphone records ambient sound can include user's voice commands.

In some embodiments, the headset device 140D may be communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data may be communicated between a headset device 140D and a server 102 in an encrypted format. In some embodiments, the vision test management platform is coupled to a global health database storing epidemiological data. The vision test management platform can be configured to cross-reference the data collected from its user accounts with the epidemiological data to identify an emerging pattern and a public health concern.

For example, a teenager's vision data may be collected and analyzed during an extended duration of time (e.g., 10 years) to identify an individual vision development trend and was cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform may integrate biometric data and global health analytics and provides a secure, personalized, and interactive environment for vision testing, which can improve precision and user experience of vision assessments and contributes to broader public health monitoring and research initiatives.

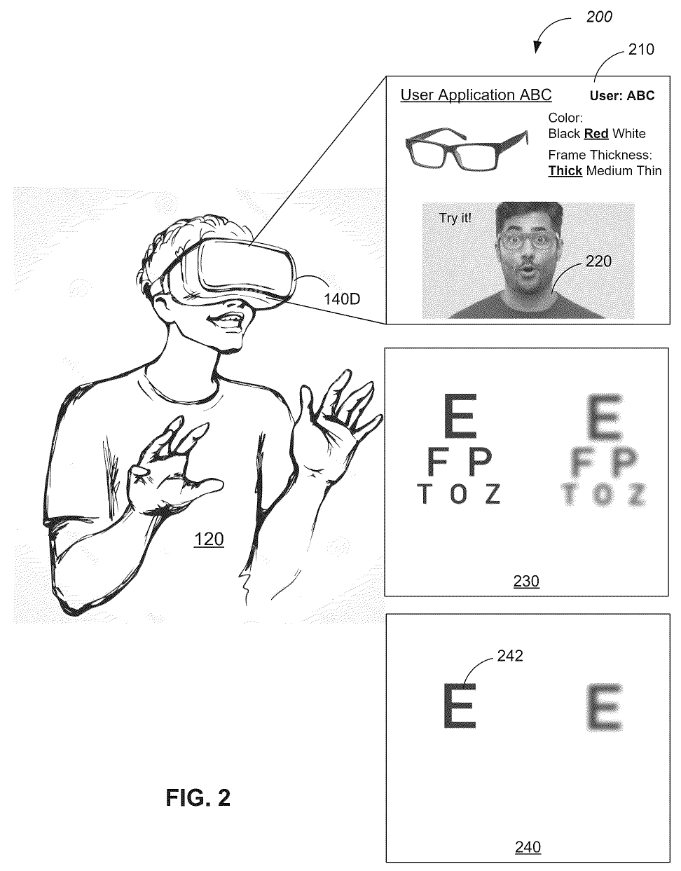

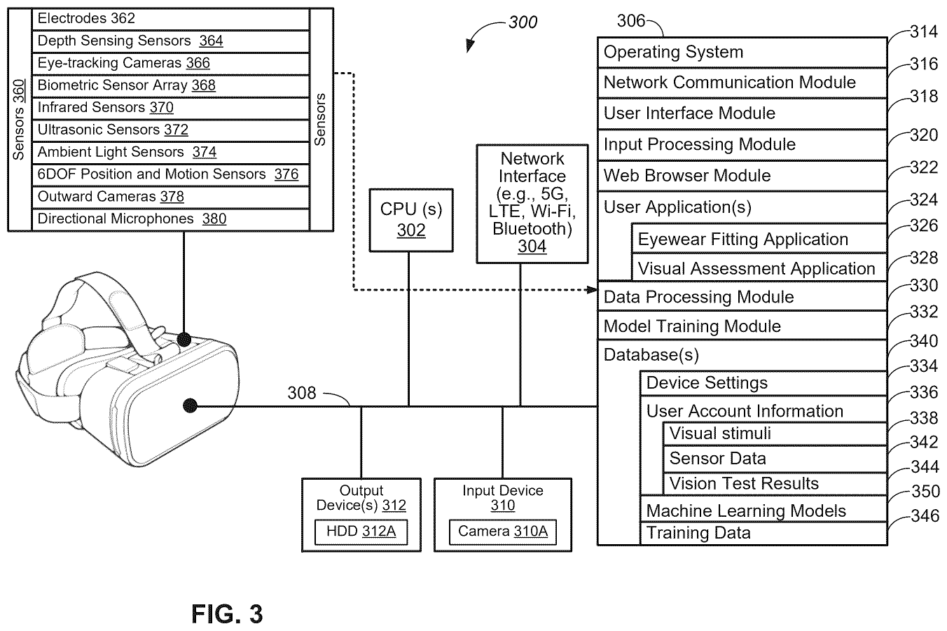

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D can include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the XR headset device 140D. In some embodiments, the microphone may record ambient sound can include user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some implementations, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 may receive, from the first user interface 210, a user feedback message indicating an issue, requesting further improvement, or confirming a fit. In some embodiments, a second user interface 230 can be displayed on a computer device 140 associated with the user 120. The second user interface 230 can include a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 can include one or more processing units (CPUs) 302, one or more network interfaces 304, memory 306, and one or more communication buses 308 for interconnecting these components (sometimes called a chipset). The computer system 300 can include one or more input devices 310 that facilitate user input, such as a keyboard, a mouse, a voice-command input unit or microphone, a touch screen display, a touch-sensitive input pad, a gesture capturing camera, or other input buttons or controls. Furthermore, in some embodiments, the computer device 140 of the computer system 300 may use a microphone for voice recognition or an eye tracking camera 366 for tracking eyeball movement. In some implementations, the computer device 140 can include one or more optical cameras (e.g., an RGB camera), scanners, or photo sensor units for capturing images. The computer system 300 may also can include one or more output devices 312 that enable presentation of user interfaces 210 and media content. The one or more output devices 312 can include one or more speakers and/or one or more visual displays.

The computer system 300 can include one or more sensors 360, which further can include one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376), one or more outward camera 378, and one or more directional microphones 380. It is noted that the one or more sensors 360 can also be included in the input device 310 and used to collect data to the computer system 300.

Memory 306 can include high-speed random-access memory, such as DRAM, SRAM, DDR RAM, or other random-access solid state memory devices; and, optionally, can include non-volatile memory, such as one or more magnetic disk storage devices, one or more optical disk storage devices, one or more flash memory devices, or one or more other non-volatile solid state storage devices. Memory 306, optionally, can include one or more storage devices remotely located from one or more processing units 302. Memory 306, or alternatively the non-volatile memory within memory 306, can include a non-transitory computer readable storage medium. In some implementations, memory 306, or the non-transitory computer readable storage medium of memory 306, may store the following programs, modules, and data structures, or a subset or superset thereof:

Operating system 314 including procedures for handling various basic system services and for performing hardware dependent tasks;

Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;

User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);

Input processing module 320 for detecting one or more user inputs or interactions from one of the one or more input devices 310 and interpreting the detected input or interaction;

Web browser module 322 for navigating, requesting (e.g., via HTTP), and displaying websites and web pages thereof can include a web interface for logging into a user account associated with a computer device 140 or another electronic device, controlling the computer device if associated with the user account, and editing and reviewing settings and data that are associated with the user account;

One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);

Data processing module 330 for processing data associated with the user applications 324, e.g., using machine learning models 350;

Model training Module 332 for obtaining training data 346 and training machine learning models 350; and

One or more databases 340 for storing at least data including one or more of:

Device settings 334 including common device settings (e.g., service tier, device model, storage capacity, processing capabilities, communication capabilities, etc.) of the computer system 300;

User account information 336 for the one or more user applications 324, e.g., user names, security questions, account history data, user preferences, and predefined account settings, where in some embodiments, the user account information 336 can include facial measurements and one or more virtual fitting parameters associated with associated with a user account of an eye fitting application 326, and in some embodiments, the user account information 336 can include visual stimuli 338, sensor data 342, and vision test results 344 associated with a user account of a visual assessment application 328; and

Machine learning models 350 including parameters (e.g., weights, biases) used to implement vision test or select eyewear for eyewear users.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in some embodiments. In some embodiments, memory 306, optionally, stores a subset of the modules and data structures identified above. Furthermore, memory 306, optionally, stores additional modules and data structures not described above.

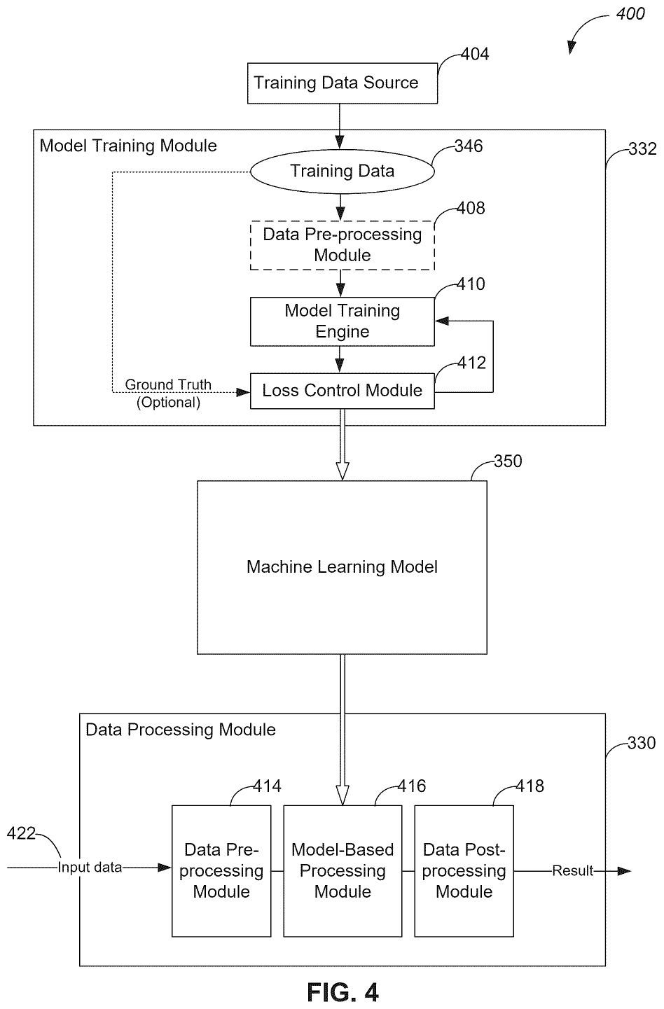

FIG. 4 is a block diagram of a machine learning system 400 for training and applying machine learning models 350 (e.g., for glass making), in accordance with some embodiments. The machine learning system 400 can include a model training module 332 establishing one or more machine learning models 350 and a data processing module 330 for processing input data 422 using the machine learning model 350. In some embodiments, both the model training module 332 and the data processing module 330 may be located within a computer device 140 (e.g., a VR headset), while a training data source 404 provides training data 346 to the computer device 140. In some embodiments, the training data source 404 can include the data obtained from the computer device 140 itself, from a server 102, from storage 106, or from another electronic device or computer device 140. Alternatively, in some embodiments, the model training module 332 may be located at a server 102, and the data processing module 330 may be located in a computer device 140. The server 102 can train the machine learning model 350 and provide the trained models 350 to the computer device 140 to process real-time input data 422 detected by the computer device 140. In some embodiments, the training data 346 provided by the training data source 404 can include a standard dataset widely used to train machine learning models 350. The input data 422 further can include sensor data. Further, in some embodiments, a subset of the training data 346 may be modified to augment the training data 346. The subset of modified training data may be used in place of or jointly with the subset of training data 346 to train the machine learning models 350.

In some embodiments, the model training module 332 can include a model training engine 410, and a loss control module 412. Each machine learning model 350 may be trained by the model training engine 410 to process corresponding input data 422 and implement a respective task. Specifically, the model training engine 410 may receive the training data 346 corresponding to a machine learning model 350 to be trained and process the training data to build the machine learning model 350. In some embodiments, during this process, the loss control module 412 can monitor a loss function comparing the output associated with the respective training data item to a ground truth of the respective training data item. In these embodiments, the model training engine 410 may modify the machine learning models 350 to reduce the loss, until the loss function satisfies a loss criterion (e.g., a comparison result of the loss function is minimized or reduced below a loss threshold). The machine learning models 350 may thereby be trained and provided to the data processing module 330 of a computer device 140 to process real-time input data 422 from the computer device 140.

In some embodiments, the model training module 402 may further can include a data pre-processing module 408 configured to pre-process the training data 346 before the training data 346 is used by the model training engine 410 to train a machine learning model 350. For example, an image pre-processing module 408 is configured to format patients' eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 can use supervised learning in which the training data 346 may be labelled and can include a desired output for each training data item (also called the ground truth, in some embodiments). In some embodiments, the desirable output may be labelled manually by people or automatically by the model training model 332 before training. In some embodiments, the model training module 332 may use unsupervised learning in which the training data 346 is not labelled. The model training module 332 is configured to identify previously undetected patterns in the training data 346 without pre-existing labels and with little or no human supervision. Additionally, in some embodiments, the model training module 332 may use partially supervised learning in which the training data is partially labelled.

In some embodiments, the data processing module 330 can include a data pre-processing module 414, a model-based processing module 416, and a data post-processing module 418. The data pre-processing modules 414 may pre-process input data 422 based on the type of the input data 422. In some embodiments, functions of the data pre-processing modules 414 arc consistent with those of the pre-processing module 408. The data pre-processing modules 414 can convert the input data 422 into a predefined data format that is suitable for the inputs of the model-based processing module 416. The model-based processing module 416 may apply the trained machine learning model 350 provided by the model training module 332 to process the pre-processed input data 422. In some embodiments, the model-based processing module 416 can also monitor an error indicator to determine whether the input data 422 has been properly processed in the machine learning model 350. In some embodiments, the processed input data may be further processed by the data post-processing module 418 to create a preferred format or to provide additional information that can be derived from the processed input data. The data processing module 330 may use the processed input data to make eyewear glasses for a patient user.

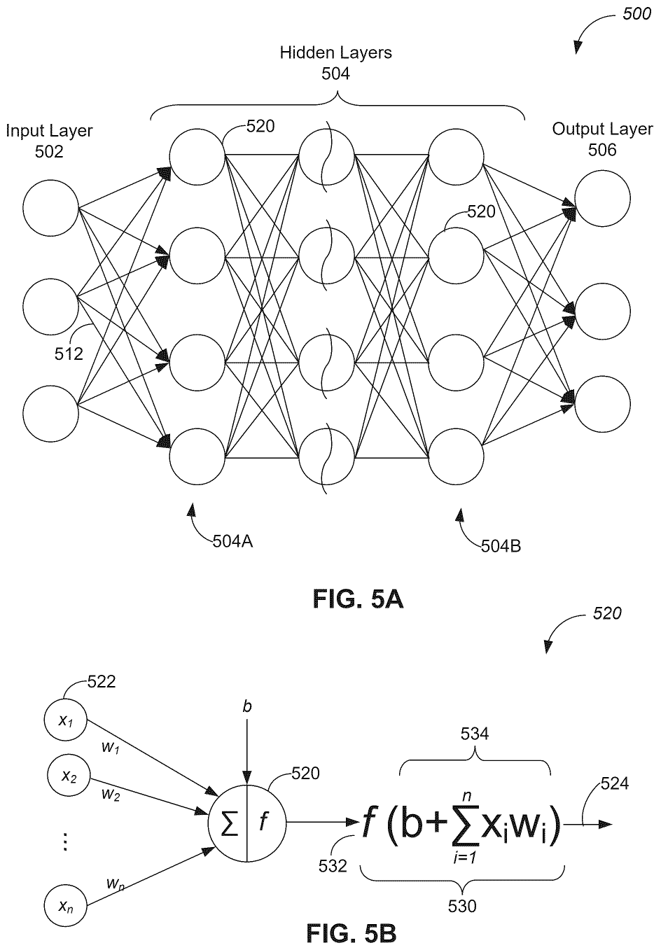

FIG. 5A is a structural diagram of an example neural network 500 applied to process input data in a machine learning model 350, in accordance with some embodiments. Further, FIG. 5B is an example node 520 in the neural network 500, in accordance with some embodiments. It should be noted that this description is used as an example only, and other types or configurations may be used to implement the embodiments described herein. The machine learning model 350 may be established based on the neural network 500. A corresponding model-based processing module 416 may apply the machine learning model 350 including the neural network 500 to process input data 422 that has been converted to a predefined data format. The neural network 500 can include a collection of nodes 520 that may be connected by links 512. Each node 520 may receive one or more node inputs 522 and applies a propagation function 530 to generate a node output 524 from the one or more node inputs. As the node output 524 is provided via one or more links 512 to one or more other nodes 520, a weight w associated with each link 512 may be applied to the node output 524. Likewise, the one or more node inputs 522 may be combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 may be organized into layers in the neural network 500. In general, the layers can include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer may only be connected with its immediately preceding and/or immediately following layer. In some embodiments, a layer may be a “fully connected” layer because each node in the layer is connected to every node in its immediately following layer. In some embodiments, a hidden layer 504 can include two or more nodes that may be connected to the same node in its immediately following layer for down sampling or pooling the two or more nodes. In particular, max pooling may use a maximum value of the two or more nodes in the layer for generating the node of the immediately following layer.

In some embodiments, a convolutional neural network (CNN) may be applied in a machine learning model 350 to process input data. The CNN employs convolution operations and belongs to a class of deep neural networks. The hidden layers 504 of the CNN can include convolutional layers. Each node in a convolutional layer may receive inputs from a receptive area associated with a previous layer (e.g., nine nodes). Each convolution layer may use a kernel to combine pixels in a respective area to generate outputs.

For example, the kernel may be to a 3×3 matrix including weights applied to combine the pixels in the respective area surrounding each pixel. Video or image data can be pre-processed to a predefined video/image format corresponding to the inputs of the CNN. In some embodiments, the pre-processed video or image data may be abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data may be processed by the data processing module 330, and two or more types of neural networks (e.g., both a CNN and an RNN) may be applied in the same machine learning model 350 to process the input data jointly.

The training process is a process for calibrating all of the weights wᵢ for each layer of the neural network 500 using training data 346 that is provided in the input layer 502. The training process typically can include two steps, forward propagation and backward propagation, which may be repeated multiple times until a predefined convergence condition is satisfied. In the forward propagation, the set of weights for different layers may be applied to the input data and intermediate results from the previous layers. In the backward propagation, a margin of error of the output (e.g., a loss function) is measured (e.g., by a loss control module 412), and the weights may be adjusted accordingly to decrease the error. The activation function 532 can be linear, rectified linear, sigmoidal, hyperbolic tangent, or other types. In some embodiments, a network bias term b may be added to the sum of the weighted outputs 534 from the previous layer before the activation function 532 is applied. The network bias b may provide a perturbation that helps the neural network 500 avoid over fitting the training data. In some embodiments, the result of the training can include a network bias parameter b for each layer.

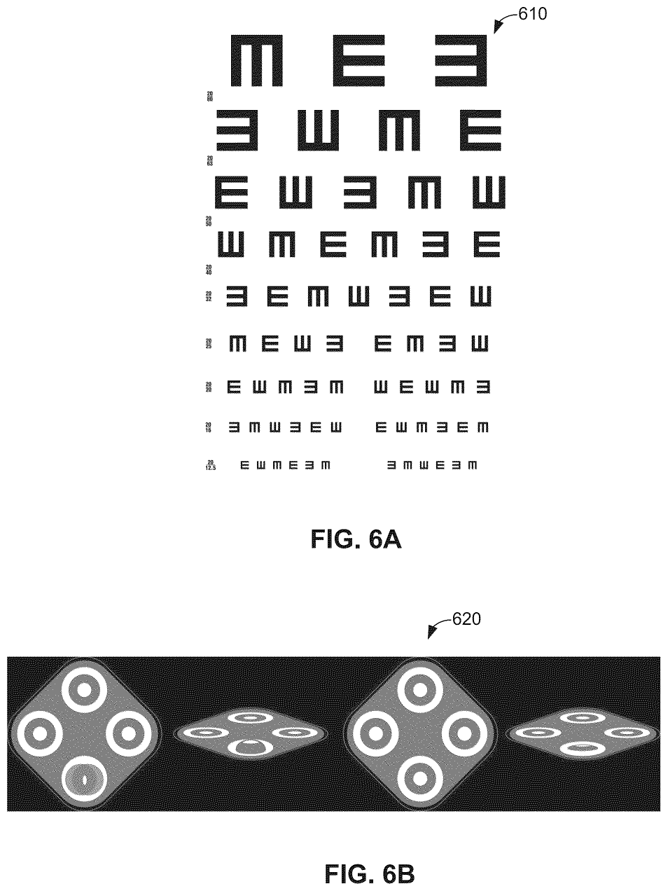

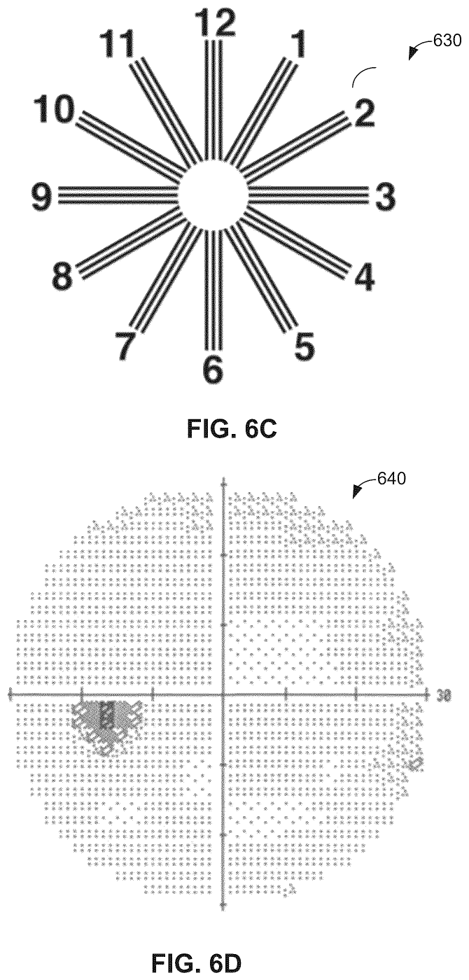

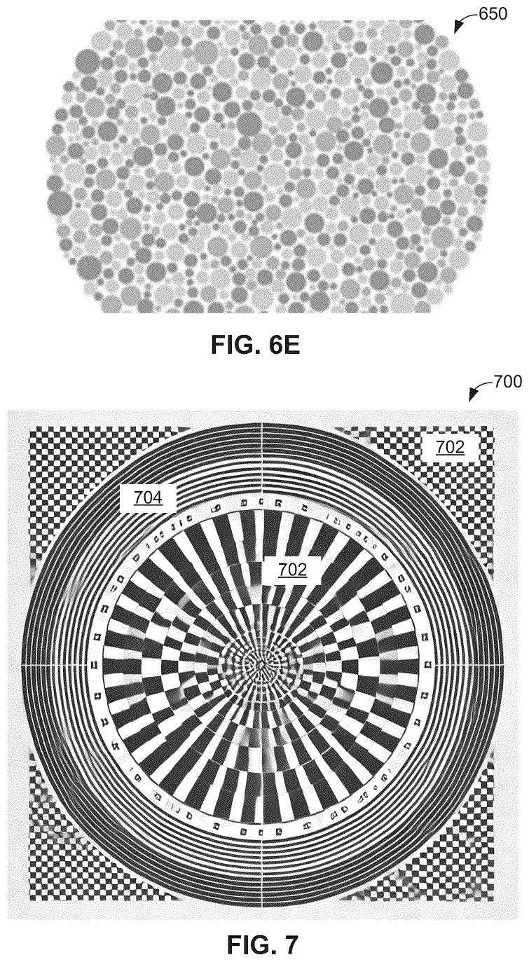

In some embodiments of the present disclosure, a vision test is implemented in a headset device 140D configured to display a user interface creating a three-dimensional (3D) virtual environment. Examples of a vision test implemented in the 3D virtual environment can include, but are not limited to a visual acuity test, a visual field test, a visual depth test, a color blindness test, a retinoscopy, a test for stereopsis, a refraction test, an astigmatism test, and a contact lens exam. FIG. 6A is an example “tumbling E” chart 610 applied in a visual acuity test, in accordance with some embodiments. FIGS. 6B, 6C, 6D, and 6E are example patterns 620, 630, 640, and 650 applied in an astigmatism test, a stereopsis test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 can include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

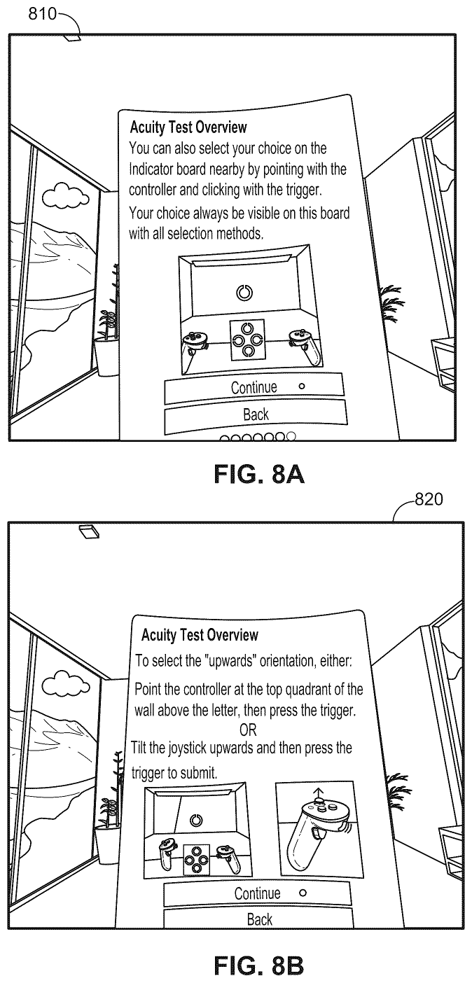

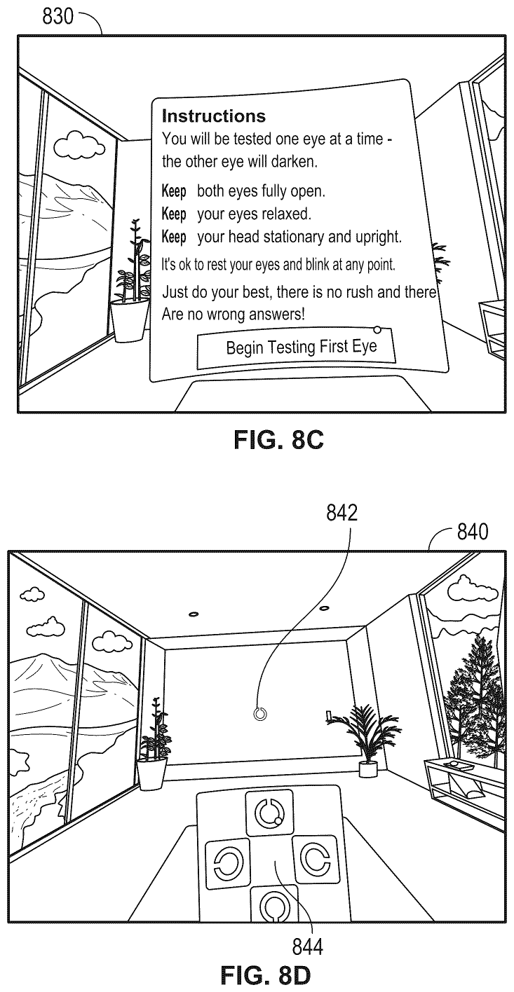

FIGS. 8A-8D can include four diagrams of example graphical user interfaces 810, 820, 830, and 840 rendered to determine a visual acuity score in a virtual environment created by a headset device 140D, in accordance with some embodiments. The user interface 810 may display an information page including instructions on controlling a headset device 140D to select one of a plurality of optotype candidates to match a target optotype displayed in the virtual environment. The user interface 820 may display an information page including two optional ways of using the controller to select the one of the plurality of optotype candidates. The user interface 830 may display an information page including general guidelines on a visual acuity assessment process. The user interface 840 may display an optotype 842 that is projected on a screen that has a first distance L1 from a user's position in the virtual environment. In a second distance L2 near the user, a selection panel 844 including a plurality of optotype candidates may be displayed, prompting the user to select one of the optotype candidates that matches the optotype 842. In some embodiments, in response to a user selection of the one of the optotype candidates, the optotype 842 displayed in the first distance L1 may be updated with a new optotype 842. Further, in some embodiments, the new optotype 842 may spin at a fast rate for a shortened duration of time (e.g., 2 seconds), before it settles in place of the original optotype 842. In an example, the optotype 842 may spin and gradually shrink in size during the shortened duration of time.

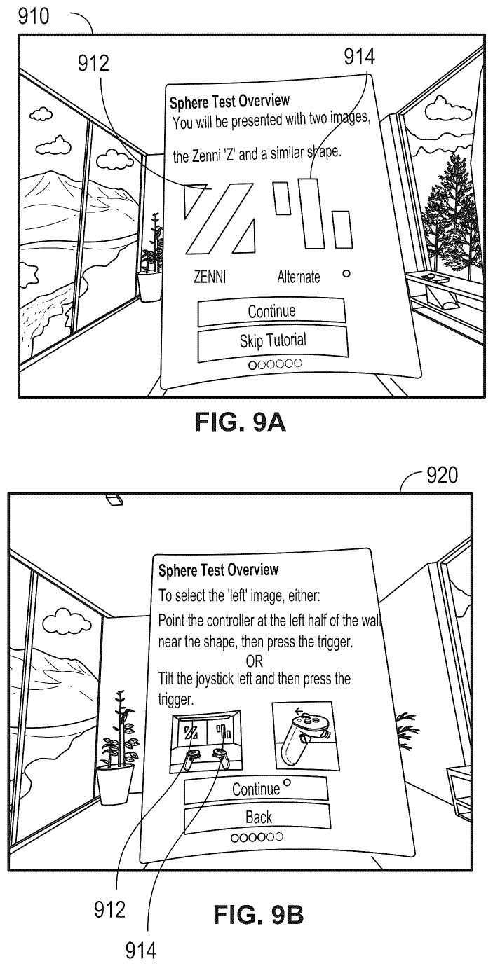

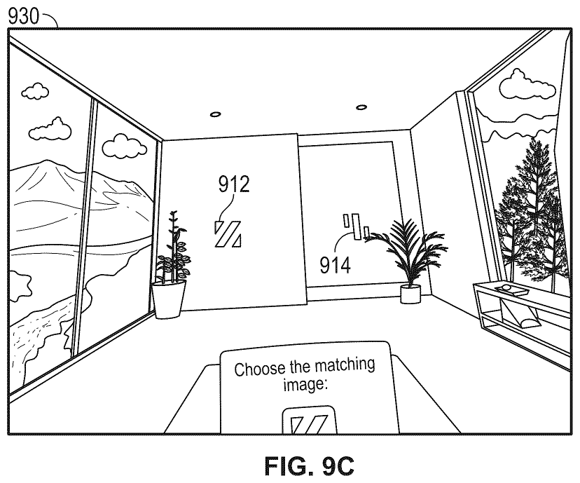

FIGS. 9A-9C can include three diagrams of example graphical user interfaces 910, 920, and 930 rendered to determine a nearsighted or farsighted power in a virtual environment created by a headset device 140D, in accordance with some embodiments. The user interface 910 may display an information page explaining that two target optotypes 912 and 914 may be displayed in the virtual environment. The user interface 920 may display an information page including two optional ways of using the controller to select one of the two target optotypes 912 and 914. The user interface 930 may display two target optotypes 912 and 914 that may be projected on a screen that has a first distance L1 from a user's position in the virtual environment. In this example, the target optotype 912 located on the left is highlighted (e.g., by being displayed in a colored background). In a second distance L2 near the user, a confirmation panel 932 may be displayed, prompting the user to select one of the two target optotypes 912 and 914. In some embodiments, in response to a user selection of the one of the two target optotypes 912 and 914, the two target optotypes 912 and 914 displayed in the first distance L1 may be updated with a new pair of two target optotypes 912 and 914. Further, in some embodiments, each optotype 912 or 914 may spin at a fast rate for a shortened duration of time (e.g., 2 seconds), before it settles in place of the original optotype 912 or 914. In an example, the optotype 912 or 914 may spin and gradually shrink in size during the shortened duration of time.

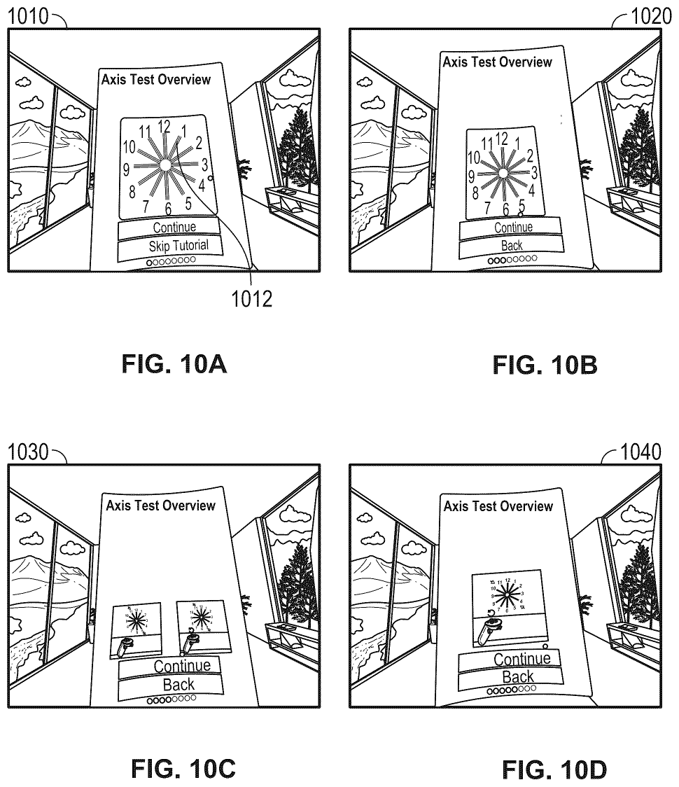

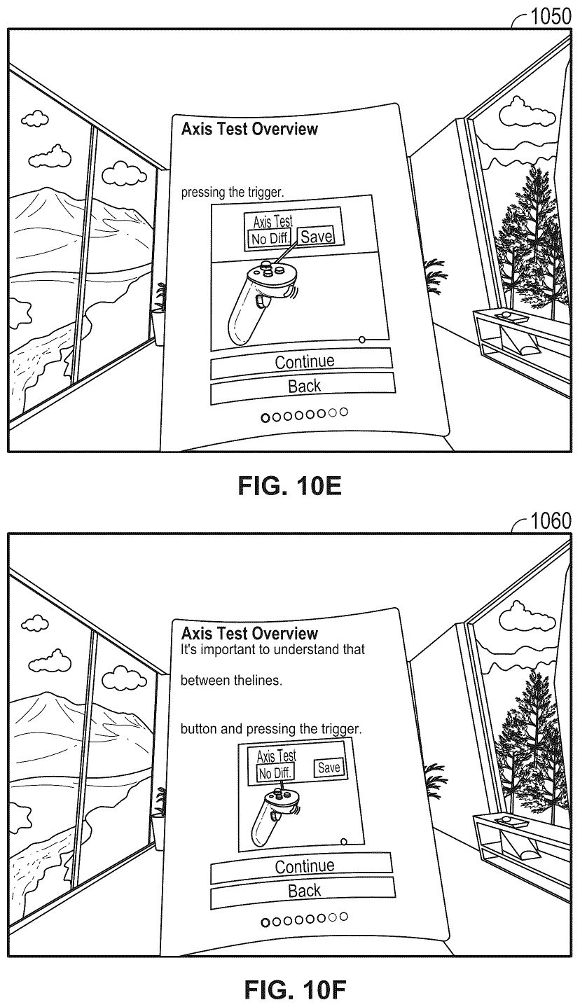

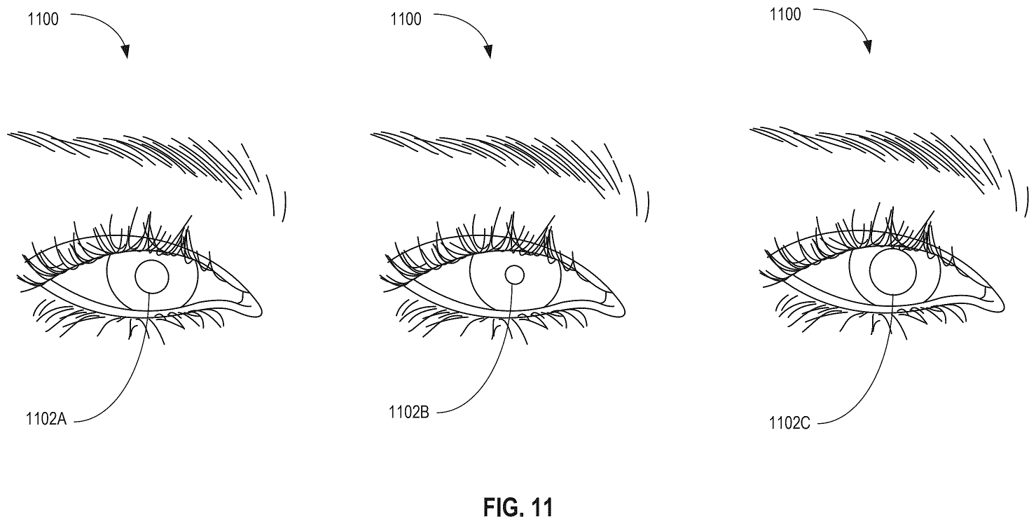

FIGS. 10A-10F can include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 may display an information page explaining that a clock diagram of converging numbered lines 1012 (which is a type of optotype) is displayed in the virtual environment. For example, the user interface 1010 can include a message, e.g., “You will be presented with a clock diagram of converging numbered lines.”

The user interface 1020 may display an information page explaining what is selected on the clock diagram of converging numbered lines 1012 displayed in the virtual environment. For example, the user interface 1010 can include a message, e.g., “Your task is to identify if any of these sets of lines appear clearer, crisper, or darker than other.”

Further, the user interface 1030 may display an information page including two optional ways of using the controller to select lines on the clock diagram of converging numbered lines 1012. For example, the user interface 1010 can include a message, e.g., “Make a selection by either pointing the controller at the lines on the clock, then pressing the trigger” and “Rotating the joystick to move the indicator arrows around the clock.”

The user interface 1040 may display an information page illustrating an embodiment having equally clear lines on the clock diagram of converging numbered lines 1012. For example, the user interface 1010 can include a message, e.g., “If two sets of neighboring lines seem to both stand out as equally clear, you can move the indicator arrows to a halfway point between those lines.”

Referring to FIG. 10E, the user interface 1050 may display an information page including an instruction using the controller to submit a selection. For example, the user interface 1010 can include a message, e.g., “After selecting a set of lines, submit your choice with the ‘Done’ button below by pointing to the controller at the button and pressing the trigger.”

Further, referring to FIG. 10F, the user interface 1060 may display an information page including an instruction using the controller to indicate that no difference is observed on the clock diagram of converging numbered lines 1012. For example, the user interface 1010 can include a message, e.g., “It's important to understand that not everybody will see a difference between the lines” and “In this case, simply select ‘No Difference’ below, by positioning the controller at the button and pressing the trigger.”

### Assessing Rapid Focusing Ability Through Gamified Tasks

Focusing ability, which can also be referred to as an amplitude of accommodation or an accommodative ability, refers to a person's ability to effectively transition between focusing on objects that are nearby and objects that are far away—and vice versa. A VR system for assessing focusing ability can include a VR headset in electronic communication with a computing device.

The VR headset is worn by a patient whose focusing ability is being assessed and can include screens, eye-tracking sensors, and eye-tracking cameras. The screens are configured to display the virtual, three-dimensional environment for the patient. The screens are also configured to display objects in the virtual environment at various distances from the patient. The eye-tracking sensors and the eye-tracking cameras are configured to collect focusing data as the patient interacts with the objects in the virtual environment. Specifically, the eye-tracking cameras and the eye-tracking sensors are configured to monitor the pupil size and the eye position of the patient.

FIG. 11 illustrates a normal pupil, a constricted pupil, and a dilated pupil, as perceived by a VR headset, in accordance with some embodiments. As shown, the eye 1100A has a pupil 1102A in a natural, relaxed state. When the patient is focusing on an object that is close to her, her pupil will constrict or shrink. As a result, the eye-tracking sensors and eye-tracking cameras of the VR headset may perceive that the patient's eye 1100 has a constricted pupil 1102B when the object in the virtual environment is close to the patient. On the other hand, when the patient is focusing on an object that is far away from her in the virtual environment, her pupil will dilate or become larger. Consequently, the eye-tracking sensors and eye-tracking cameras of the VR headset may perceive that the patient's eye 1100 has a dilated pupil 1102C when the object in the virtual environment is far away from the patient.

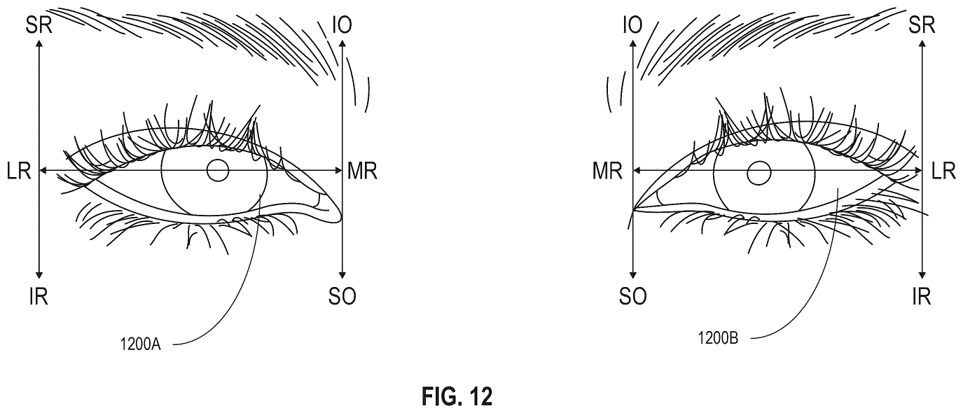

FIG. 12 illustrates charts of the cardinal gaze positions labeled with the extraocular muscles that correspond with the gaze positions superimposed over the patient's eyes, in accordance with some embodiments. FIG. 12 shows charts 1202A and 1202B over the right eye 1200A and the left eye 1200B, respectively. The charts 1202A, 1202B are an “H” diagram of the different directions in which the eyes 1200A, 1200B can look, and the charts 1202A, 1202B are labeled with the extraocular muscles that correspond with those directions.

Every person must use different eye muscles to move her eyes in different directions. Specifically, the superior rectus (SR) is a muscle on top of the eye that moves the eye upward; the inferior rectus (IR) is a muscle on the bottom of the eye that moves the eye downward; the medial rectus (MR) is a muscle on the portion of the eye that is near the nose and moves the eye inward (toward the nose); the lateral rectus (LR) is a muscle on the portion of the eye that is near the car and moves the eye outward (toward the car); the superior oblique (SO) is a muscle that starts at the back of the eye socket, passes by the nose, and attaches to the top of the eye to rotate the eye inward, move the eye downward, and move the eye outward; and the inferior oblique (IO) is a muscle that starts at the front of the eye socket near the nose and attaches to the bottom of the eye to rotate the eye outward, move the eye upward, and move the eye outward. Each of these muscles is labeled in the charts in FIG. 12.

When the patient is focusing on an object that is close to her, her eyeballs 1200A, 1200B will turn in towards her nose, which is an eye movement that largely relies on the medial rectus. When the object is close to the patient in the virtual environment, the eye-tracking sensors and the eye-tracking cameras will expect to perceive the eyeballs 1200A, 1200B turning in towards each other. In contrast, when the patient is focusing on an object that is far away from her, her eyeballs 1200A, 1200B will turn out towards her cars, which largely involves use of the lateral rectus muscles. When the object is far away from the patient in the virtual environment, the eye-tracking sensors and the eye-tracking cameras of the VR headset will expect to perceive the eyeballs 1200A, 1200B turning out towards the patient's ears (i.e., towards the “LR” portion of the chart).

Optionally, the eye-tracking sensors and cameras are infrared sensors and cameras. Optionally, there is at least one eye-tracking camera pointed at each pupil of the patient when the patient wears the VR headset.

The VR system also can include a computing device, which is configured to cause a virtual, three-dimensional environment and objects within that environment to be displayed on screens of the VR headset. The objects can be displayed at various positions in the virtual environment, at various distances from the patient. In some embodiments, an algorithm determines the various distances at which the objects are displayed.

For example, the algorithm might quickly move the objects between positions that are close to the patient and positions that are far away from the patient to challenge the patient and identify the outer limits of the patient's focusing ability. Optionally, the algorithm might display the objects at distances that the patient struggles to focus on in order to gather more information about the patient's problem areas, which would lead to a narrower and more informative evaluation.

The computing device can also be configured to prompt the patient to focus her gaze on objects in the virtual environment and receive the focusing data from the VR headset while the patient focuses her gaze on the objects in the virtual environment. In some embodiments, the computing device can include an algorithm that processes the focusing data to evaluate a speed at which the patient can change her depth of focus.

Optionally, the computing device can include an algorithm that processes the focusing data to evaluate an accuracy with which the patient focuses on different objects in the virtual environment. In some embodiments, the eye-tracking sensors and the eye-tracking cameras are configured to quantify the size of the patient's pupil and communicate these measurements to the computing device, which can be configured to process these measurements and calculate the patient's depth of focus. Similarly, the eye-tracking sensors and the eye-tracking cameras are configured to quantify the position of the patient's eyeballs and communicate these measurements to the computing device, which can be configured to process these measurements and calculate the patient's depth of focus. With this information, the computing device can calculate the position, including the depth of focus, of the patient's gaze. As a result, the computing device can calculate the patient's focusing accuracy by comparing the patient's gaze position to the position of the object in the virtual environment, which the computing device knows because the computing determines the position of the objects in the virtual environment. Generally, a patient with a healthy focusing ability will have a focusing accuracy of approximately 97%, at least. Smaller focusing accuracies can be indicative of ocular conditions such as strabismus and phoria.

In some embodiments, the computing device can include an algorithm that processes the focusing data to evaluate a focusing consistency of the patient. When a patient has a high focusing consistency, she can maintain a healthy focusing accuracy over an extended period of time. A patient can have a high focusing consistency if she can maintain a focusing accuracy of approximately 97% at least over a period of approximately one hour. A patient can also have a high focusing consistency if she can maintain an extremely high focusing accuracy over a shorter period of time (e.g., a focusing accuracy of 99% over a period of ten minutes).

In some embodiments, the computing device can include an algorithm that processes the focusing data in real-time as the patient follows the prompts and focuses on the various objects in the virtual environment.

In other embodiments, the computing device can include an algorithm that is configured to process the focusing data to identify and measure deficiencies in the patient's focusing ability in order to evaluate the patient for accommodative dysfunction and presbyopia.

Optionally, a handheld device (e.g., a controller, a VR handset, or a glove) is in electronic communication with the VR headset and the computing device. The patient can use the handheld device to interact with the objects in the virtual environment. For example, the patient can also use the handheld device to catch or shoot objects in the virtual environment.

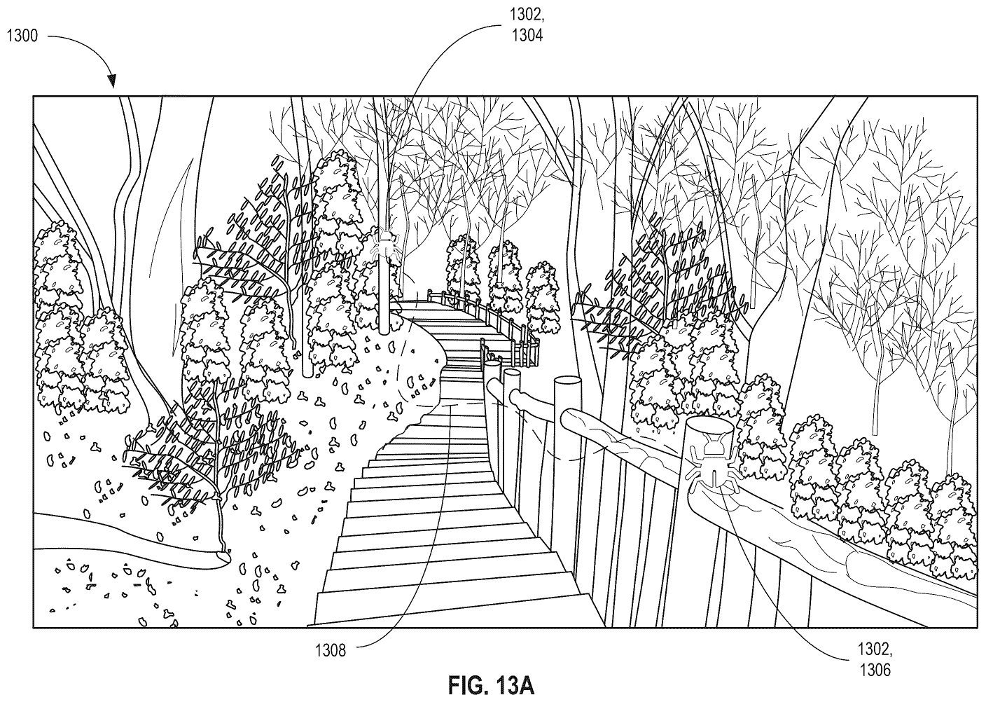

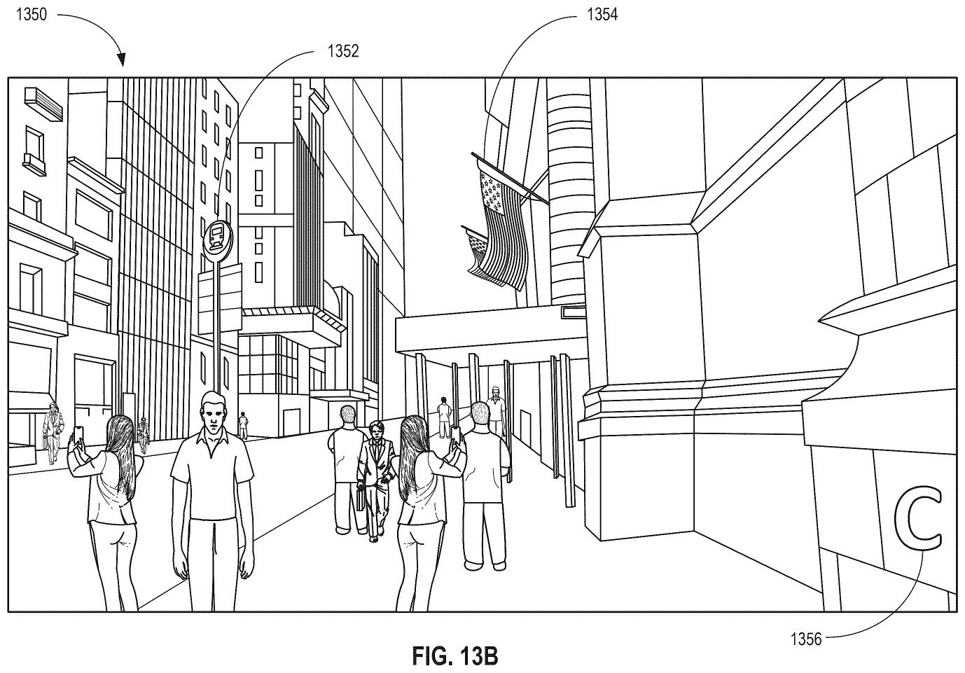

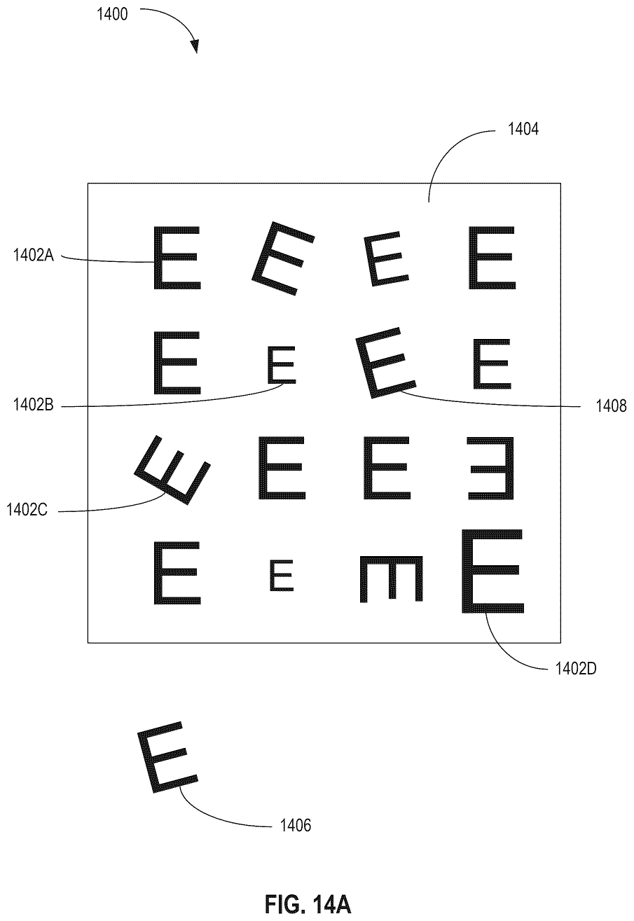

The rapid near/far focusing ability assessment can begin after the patient dons the VR headset. The computing device causes a virtual environment and objects in the virtual environment to be displayed on the screens of the VR headset. FIG. 13A illustrates a virtual environment that can include an object that can be positioned at different distances from the patient, in accordance with some embodiments. Specifically, FIG. 13A illustrates a virtual environment 1300 of a botanical garden, which the computing device can cause to be displayed on the screens of the VR headset. The computing device can also cause an object, such as a bug 1302, to be displayed in the virtual environment 1300. For example, the bug 1302 can be displayed at a position 1304 that is far away from the patient in the virtual environment 1300.

The computing device causes the bug 1302 to be displayed at a first position 1304 in the virtual environment 1300 that is far away from the patient and then prompts the patient to focus on the bug 1302 at the first position 1304. While the patient attempts to focus on the bug 1302 at the first position 1304, the eye-tracking sensors and the eye-tracking cameras monitor the patient's eyes to quantify the patient's eye position and pupil size (i.e., the first eye position and the first pupil size), as described above.

The computing device then causes the bug 1302 to be displayed at a second position 1306 in the virtual environment 1300 that is near the patient. In some embodiments, the first position 1304 is closer to the patient than the second position 1306. Optionally, the computing device can change an amount of time (i.e., milliseconds, seconds, minutes, etc.) between the time when the bug 1302 is displayed at the first position 1304 and the time when the bug 1302 is displayed at the second position 1306. When the computing device increases the amount of time between displaying the bug 1302 at the first and second positions 1304, 1306, the patient can adjust her focus at a more leisurely pace. On the other hand, when the computing device decreases the amount of time, the patient must adjust her focus more quickly.

The computing device prompts the patient to focus on the bug 1302 at the second position 1306. When the patient attempts to focus on the bug 1302 at the second position 1306, the eye-tracking sensors and the eye-tracking cameras monitor the patient's eyes to quantify the patient's eye position and pupil size (i.e., the second eye position and the second pupil size).

Optionally, the eye-tracking sensors and the eye-tracking cameras monitor the patient's eyes in real-time while the patient tracks the object in the virtual environment or otherwise interacts with the virtual environment during the assessment. By monitoring the patient's eyes in real time, the computing device can process the patient's focusing ability in real-time and adjust the difficulty of the assessment based on the patient's focusing ability.

For example, if the patient is struggling during the assessment and demonstrating poor focusing ability, the computing device can decrease the difficulty of the assessment (e.g., by decreasing the distance between the first position and the second position of the object). Not only does this make the assessment less frustrating for the patient, which limits the likelihood that the patient will stop engaging with the assessment, but decreasing the difficulty in accordance with the patient's focusing ability increases the accuracy of the assessment by homing in on the limitations of the patient's focusing ability. Optionally, if the patient has trouble transitioning between the first eye position and the second eye position, then the computing device can repeat that motion to gather more data, which will help determine why the patient has trouble with that transition. Conversely, if the patient easily transitions between the first pupil size and the second pupil size, then the computing device does not need to repeat this motion because the patient has exhibited healthy behavior that does not need further examination.

In some embodiments, the computing device causes the bug 1302 to move from the first position 1304 to the second position 1306 along a path 1308. The path 1308 can be curved and winding as shown in FIG. 13A. In some embodiments, the path 1308 is a straight line. In other embodiments, the path 1308 has a jagged and randomized trajectory. Other paths are also possible. Optionally, the bug 1302 is shown at the first position 1304, is removed from the virtual environment 1300, and then reappears at the second position 1306.

In some embodiments where the computing device moves the object towards the patient, the computing device can also prompt the patient to catch the object when the object reaches the second position or when the object reaches the patient. Optionally, the computing device moves the object away from the patient and prompts the patient to run after the object and catch it. In other embodiments, the computing device can prompt the patient to shoot the object at either the first or second position. All three of these options blend the patient's visual and muscular functions in order to assess the patient's hand-eye coordination. Use of the VR system is particularly beneficial in this context because number of positions at which the object can be displayed and the number of combinations of near and far are limitless. This facilitates the collection of a huge dataset, which leads to very narrow and accurate assessments of rapid near/far focusing ability.

The computing device can also be configured to calculate the time taken by the patient to transition between near and far focal depths by calculating the time taken by the patient to transition between different eye positions and/or pupil sizes. If the patient has an ideal near/far focusing ability (e.g., is in the top 1% of focusing efficiency), her eyes will move quickly and her pupil size will change rapidly in response to changing the depth of focus of her gaze.

For example, the patient can change her gaze from looking at an object 20 feet away to looking at an object 16 inches away in under 1 second. On the other hand, if the patient has a degraded focusing ability, she might make this transition from 20 feet to 16 inches in approximately 2-3 seconds or more. Optionally, the computing device calculates the focus time of the patient in real-time as the patient completes the assessment. In other embodiments, the computing device compares the focus time to a database of focus times from individuals with known focusing abilities to assess the patient's focusing ability.

In some embodiments of the VR assessment method, the computing device displays a plurality of objects in the virtual environment. Each object in the virtual environment has a different characteristic and is positioned at a different distance from the patient. FIG. 13B illustrates a virtual environment that can include multiple objects positioned at different distances from the patient, in accordance with some embodiments. In particular, FIG. 13B illustrates a virtual environment 1350 of a busy city street. The virtual environment 1350 can include multiple objects such as a bus sign 1352, a flag 1354, and an optotype 1356, each of which has a different characteristic and is positioned at a different distance from the patient.

For example, the flag 1354 can be displayed centrally in the patient's field of vision, and the optotype 1356 is displayed peripherally in the patient's field of vision. Additionally, the bus sign 1352 is quite far away from the patient because it is across the street, but the optotype 1356 is very close to the patient. Moreover, the bus sign 1352, the flag 1354, and the optotype 1356 can all be different colors, have different sizes, have different shapes, and be displayed at different contrasts or brightnesses relative to the rest of the virtual environment.

Optionally, the virtual environment is a maze, and the computing device causes multiple objects to be displayed at each juncture or turn of the maze. To navigate through the maze, the patient must focus her gaze on the correct object in accordance with a prompt from the computing device (e.g., “focus on the red circle” or “focus on the upright ‘E’”).

The computing device prompts the patient to focus on different objects in the virtual environment, and the eye-tracking sensors and the eye-tracking cameras monitor patient's eyes to quantify the patient's eye positions and pupil sizes while the patient focuses on the different objects, as described above. Optionally, the eye-tracking sensors and the eye-tracking cameras monitor the patient's eyes in real-time while the patient completes the assessment, and the computing device processes the patient's eye positions and pupil sizes in real-time.

In some embodiments, the computing device can change an amount of time between prompting the patient to focus on different objects, as described above.

The computing device can also calculate a focus time of the patient, as described above.

In some embodiments, the computing device generates a report summarizing the patient's eye movement patterns, diagnosing eye movement disorders, listing treatment and evaluation recommendations, and predicting future eye movement disorders. This report can be accessed by a physician, the patient, or the patient's caretaker at a user interface in the computing device.

### Training Ocular Muscles Through Targeted Exercise

As described above, a person must use different eye muscles to move her eyes in different directions. These muscles are the superior rectus (SR), the inferior rectus (IR), the medial rectus (MR), the lateral rectus (LR), the superior oblique (SO), and the inferior oblique (IO). The direction associated with each of these muscles is described above and is illustrated in FIG. 12. Each of these muscles can be targeted and individually trained using different eye exercises, which is valuable for correcting eye misalignment, improving symptoms of nystagmus, preventing the degradation of focusing ability, and even adjusting a patient's front-facing appearance (e.g., how the patient's eyes are positioned).

A VR system for administering eye exercises that are targeted at different ocular muscles can include a VR headset in electronic communication with a computing device. The VR headset is worn by a patient and can include screens, eye-tracking sensors, and eye-tracking cameras. The screens are configured to display a virtual, three-dimensional environment. The eye-tracking sensors and the eye-tracking cameras are configured to collect information about the patient's eye movements as the patient completes the eye exercises. In some embodiments, the eye-tracking sensors are configured to collect the eye movement data in real-time and communicate the eye movement data to the computing device in real-time. Optionally, the eye-tracking sensors and cameras are infrared sensors and cameras. Optionally, there is at least one eye-tracking camera pointed at each pupil of the patient when the patient wears the VR headset.

In some embodiments, the VR headset can include speakers that are configured to provide audio feedback to the patient as the patient participates in the eye exercises. For example, the VR headset may provide auditory prompts to the patient, beep to indicate when the patient is incorrectly completing the exercises, or provide congratulatory noises when the patient successfully completes an exercise to encourage and motivate the patient.

In other embodiments, the VR headset can include vibrating motors that are configured to provide haptic feedback to the patient as the patient participates in the eye exercises. The haptic feedback can comprise vibrations that make the virtual environment more realistic (e.g., the patient might experience vibrations when she is near a large truck) or buzzes that indicate whether the patient is correctly or incorrectly completing the exercises.

The computing device of the VR system is configured to cause a virtual environment to be displayed on the screens of the VR headset (such as the virtual environments in any of FIGS. 13A-16B). The computing device can also be configured to cause objects to move in three dimensions throughout the virtual environment. Optionally, the algorithm is configured to adapt the position and/or movement of the object in the virtual environment. In some embodiments, this algorithm processes the eye movement data collected by the VR headset to determine an extent and manner in which to change the position and/or movement of the object in the virtual environment.

For example, if the patient is easily tracking the object, then the algorithm can make the object move faster or in parts of the patient's field of vision that the patient has trouble looking at. This challenges the patient and expands the outer limits of the patient's ocular muscles, improving the patient's visual health.

The computing device can also be configured to process the eye movement data collected by the VR headset. In some embodiments, the computing device can comprise an algorithm to process the eye movement data. Optionally, the algorithm processes the eye movement data in real-time as the patient tracks the object throughout the virtual environment. In other embodiments, the algorithm is configured to process the eye movement data to evaluate a degree to which the patient is successfully completing the eye exercises.

In some embodiments, the computing device generates a report summarizing the patient's eye movement patterns, diagnosing eye movement disorders, listing treatment and evaluation recommendations, and predicting future eye movement disorders. Optionally, the report highlights eye muscles that the patient may focus on during future training sessions based on the patient's performance in the previous training sessions. The report can also recommend specific training exercises that focus on the recommended muscles. This report can be accessed by a physician, the patient, or the patient's caretaker at a user interface in the computing device.

The ocular muscle training can begin after the patient dons the VR headset, and the computing device causes a virtual environment to be displayed on the screens of the VR headset. The computing device also causes one or more objects to be displayed in the virtual environment at a first position. Optionally, the objects are circles, rings, or optotypes.

The position of each object in the virtual environment is defined by a set of three-dimensional coordinates: x, y, and z coordinates. The x coordinate corresponds to an x-axis that is left and right relative to the patient, the y coordinate corresponds to a y-axis that is up and down relative to the patient, and the z coordinate corresponds to a z-axis that is in front of and behind the patient. The three-dimensional coordinates of each object changes as each object moves throughout the virtual environment.

In some embodiments, the computing device also causes the orientation of the objects to change within the virtual environment.

Through the VR headset, the patient is prompted to track at least one of the objects in the virtual environment with her eyes. The object can move in any direction throughout the virtual environment to induce the patient to move her eyes up, down, in, out, left, right, near, and far. All of these motions exercise the different ocular muscles, which improves the patient's visual acuity and focus stabilization. The object can also move at different speeds to induce quick muscle movements and slow muscle movements. Quick muscle movements improve the patient's ability to quickly change the focal depth and position of her gaze. Meanwhile, slow muscle movements engage all of the surrounding ocular muscles without rapid twitch. The slow movements are inherently smoother, which makes monitoring the eye movements easier, forces the eyes to work together precisely, and generally trains the eye muscles in a more holistic manner that improves overall vision.

While the patient moves her eyes around as part of the eye exercises, the eye-tracking sensors and the eye-tracking cameras monitor the patient's eyes. In some embodiments, the eye-tracking sensors and eye-tracking cameras are constantly monitoring the patient's eyes-even when the patient is not moving her eyes around to view the object in different positions or to view different parts of the virtual environment.

The computing device can also adjust the difficulty of the eye exercises based on how successfully the patient is completing the eye exercises. Optionally, the computing device looks for a certain threshold of consistency in the patient's success before increasing the difficulty. For example, if the patient is doing well, the computing device can increase the difficulty (by increasing the speed of the moving object, by displaying the object in a wider field of vision, by decreasing the contrast between the object and the virtual environment, and other changes that have previously been described or referred to). In situations where the patient can handle the increased difficulty, the computing device facilitates a faster and/or greater strengthening of the patient's eye muscles. On the other hand, if the patient is not doing well, the computing device can decrease the difficulty to prevent the patient from becoming discouraged and quitting the training session.

Optionally, one of the screens in the VR headset can be shut off to occlude one of the patient's eyes during an eye exercises. This facilitates individual training of the eyes, which is particularly valuable where the patient's eye muscles have different capabilities and deficiencies.

### Vision Training Using Feedback-Adjusted Visual Challenges

A VR system for administering feedback-adjusted visual challenges to train a patient's vision can include a VR headset in electronic communication with a computing device. The VR headset is worn by the patient who is undergoing vision training and can include screens, eye-tracking sensors, and eye-tracking cameras. The screens are configured to display visual tasks in a virtual environment. The eye-tracking sensors and the eye-tracking cameras are configured to monitor the patient's eyes and communicate information about the patient's eyes during the vision training to the computing device.

Specifically, the eye-tracking sensors and eye-tracking cameras monitor the patient's eye movements and fixation points. This is meaningful because the patient might lock in on the correct fixation point during the vision training, but monitoring her eye movements can inform the computing device that the patient is taking an unnecessarily lengthy route to the fixation point. This inefficient route might suggest that the patient has issues with her eye muscles or nerves that are making the straightforward, efficient route difficult or impossible. It can also be possible that the patient may take an efficient path between two different points but never actually reach the fixation point, which suggests that the patient cannot properly focus her eyes on a target.

In some embodiments, the eye-tracking sensors are configured to collect the eye data in real-time and communicate the eye data to the computing device in real-time. Optionally, the eye-tracking sensors and cameras are infrared sensors and cameras.

In some embodiments, the VR headset can include speakers that are configured to provide audio feedback to the patient as the patient participates in the eye exercises. For example, the VR headset may provide auditory prompts to the patient, beep to indicate when the patient is incorrectly completing the exercises, or provide congratulatory noises when the patient successfully completes an exercise to encourage and motivate the patient.

In other embodiments, the VR headset can include vibrating motors that are configured to provide haptic feedback to the patient as the patient participates in the eye exercises. The haptic feedback can comprise vibrations that make the virtual environment more realistic (e.g., the patient might experience vibrations when she is near a large truck) or buzzes that indicate whether the patient is correctly or incorrectly completing the exercises.

In some embodiments, a handheld device (e.g., a controller or a VR handset) is in electronic communication with the VR headset and the computing device. The patient can use the handheld device to interact with the virtual environment displayed and the optotypes on the screens of the VR headset. For example, the patient can press a button to confirm that she is perceiving an optotype on the screen, use a controller to type in or select a brief description of the optotype, or even click on the optotype in the virtual environment.

The computing device is configured to cause a visual task in a virtual environment to be displayed on the screens of the VR headset. The computing device can also be configured to receive eye data collected by the VR headset. Optionally, the computing device can include an algorithm that processes the eye data. The algorithm can process the eye data in real-time as the patient completes the visual task.

In some embodiments, the computing device processes the eye data to calculate an amount of time taken by the patient to complete each visual task. Optionally, the computing device calculates the amount of time in real-time as the patient completes each visual task.

In other embodiments, the computing device processes the eye data to analyze an eye movement accuracy of the patient. Optionally, the computing device analyzes the eye movement accuracy in real-time as the patient completes each visual task. A patient who can obtain a fixation point that is within two degrees of a target point has a healthy eye movement accuracy.

In some embodiments, the computing device processes the eye data to analyze an eye movement speed of the patient. Optionally, the computing device analyzes the eye movement speed in real-time as the patient completes each visual task. A patient with a healthy eye movement speed has a gaze position that generally lags behind a target position by less than approximately 0.5 s.

In other embodiments, the computing device processes the eye data to analyze an eye movement stability of the patient. Optionally, the computing device analyzes the eye movement stability in real-time as the patient completes each visual task. A patient who can maintain a consistent eye movement accuracy is generally considered to have a healthy eye movement stability. For example, a patient has a healthy eye movement stability if she can repeat a gaze position that is within one degree of a target point at least five times consecutively.

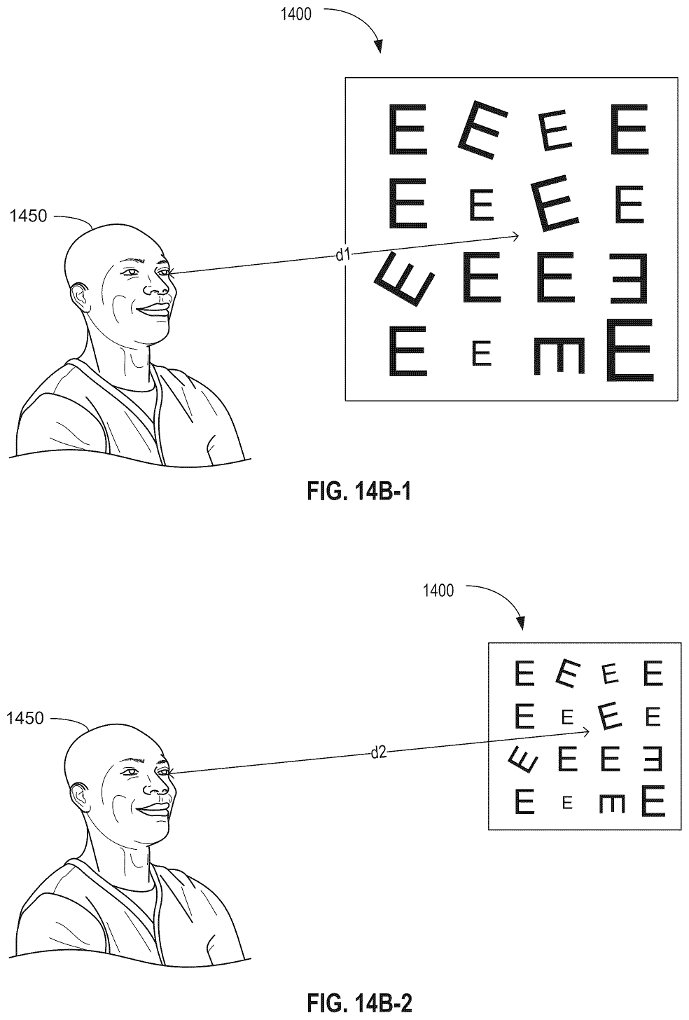

The VR method for vision training using feedback-adjusted exercises can begin after the patient dons the VR headset. The computing device causes a virtual environment to be displayed on the screens of the VR headset. In accordance with some embodiments, in the VR method for vision training using feedback-adjusted exercises, the computing device also causes an array of optotypes to be displayed in the virtual environment. For example, the computing device can cause the array of optotypes displayed in FIG. 14A to be displayed in the virtual environment, as described in greater detail below.

The array of optotypes can be symmetrical or asymmetrical. Further, the optotypes can be arranged in rows or otherwise organized in a desired pattern. For example, the array of optotypes can include optotypes stacked in a pyramid shape or optotypes scattered around the virtual environment.

FIG. 14A illustrates an array of optotypes with different contrasts relative to the virtual environment, colors, orientations, and sizes, in accordance with some embodiments. As shown, the virtual environment 1400 can include an array of optotypes 1402 displayed over a background 1404. In some embodiments, the optotypes 1402 are black and the background 1404 is white. In other embodiments, the optotypes 1402 or white and the background 1404 is black. Optionally, the background 1404 is red or green, which makes the vision training more challenging for patients with color perception deficiencies to help increase visual acuity. Other colors for the optotypes 1402 and the background 1404 are also possible.

In FIG. 14A, the optotypes 1402 have different sizes and orientations. Optotype 1402A is a mid-sized “E” in an upright orientation. Optotype 1402B is a small “E,” optotype 1402C is rotated counterclockwise, and optotype 1402D is a large “E.” Various combinations of sizes and orientations are possible.

Through the VR headset, the patient can receive a prompt to match a model optotype 1406 with a target optotype 1408 in the array of optotypes 1402. The patient can click on the target optotype 1408 with the handheld device, touch it in the virtual environment, or focus on it with her eyes. While the patient attempts to match the model optotype 1406 with the target optotype 1408, the eye-tracking sensors and the eye-tracking cameras monitor the patient's eyes. In some embodiments the eye-tracking sensors and the eye-tracking cameras continuously monitor the patient's eyes throughout the duration of the vision raining. Optionally, the eye-tracking sensors and the eye-tracking cameras monitor the patient's eyes to collect data about the eye movements of the patient to evaluate the agility of the patient's eye muscles. Optionally, the eye-tracking sensors and the eye-tracking cameras monitor the patient's eyes to collect data about the pupillary responses of the patient to evaluate the speed and effectiveness of the patient's focus adjustments. In some embodiments, the eye-tracking sensors and the eye-tracking cameras also monitor the patient's response time—that is, the amount of time taken by the patient to match the model optotype 1406 with the target optotype 1408.

Based on the patient's eye movements, pupillary responses, and response time, the computing device can change the color, brightness, contrast, size, and/or orientation of the model optotype 1406 and/or the optotypes 1402 in the array, which can include the target optotype 1408. The computing device then prompts the patient to match the changed model optotype 1406 to the target optotype 1408, the model optotype 1406 to the changed target optotype 1408, or the changed model optotype 1406 to the changed target optotype 1408.

Some embodiments of the VR method for vision testing using feedback-adjusted visual tasks can include displaying the visual task at varying distances from the patient as shown in FIGS. 14B-1 and 14B-2. The visual task can be the optotype matching task that is described above with respect to the first embodiment, but other visual tasks are also possible (e.g., tracking moving objects, targeting objects at different parts of the visual field, etc.).

FIGS. 14B-1 and 14B-2 illustrate a patient reading a vision chart at two different distances, in accordance with some embodiments. In FIG. 14B-1, the vision test 1400 is a distance d1 from the eyes of the patient 1450. In FIG. 14B-2, the vision test 1400 is a distance d2 from the eyes of the patient 1450, where the distance d2 is greater than the distance d1. Optionally, the distance d1 is greater than the distance d2. Throughout the duration of the vision test, the computing device can adjust the distances d1 and d2 between the patient 1450 and the vision test to increase or decrease the difficulty of the vision testing.

Changing the distance between the patient's eyes and the visual task can occur in three dimensions. For example, the visual task can be placed at different depths away from the patient to test focal depth. The visual task can also be placed in different areas of the patient's field of vision (e.g., central vision and peripheral vision) to test the patient's peripheral vision, saccades, and smooth pursuits. In some embodiments, the visual task is placed at randomized positions that the patient cannot predict. With an unpredictable location, the patient cannot adapt to the new visual task more easily due to preparing to complete the visual task at the second position.

In some embodiments, the computing device can also change the frequency with which the position of the visual task is changed. Generally, changing the position of the visual task more frequently is a greater challenge for patients because it requires frequent changes to the patient's gaze.

As described above, in some embodiments of the VR method, the eye-tracking sensors and the eye-tracking cameras can monitor the patient's eyes while the patient completes the visual task to collect eye data such as eye movements, pupillary responses, response times, etc.

Further, in some embodiments of the VR method, the computing device can determine the difficulty of the vision test based on the eye data collected by the eye-tracking sensors and the eye-tracking cameras. The computing device looks for a certain threshold of consistency in the patient's success before increasing the difficulty. In some embodiments, increasing the difficulty can comprise changing the color of the optotypes in the array to colors that are harder for the patient to see, which the computing device would be able to identify based on the first and second inputs. In other embodiments, increasing the difficulty can comprise moving the visual task closer to a far-sighted patient or farther away from a near-sighted patient.

### Assessing the Impact of Environmental Factors on Vision Through Simulated Exposure

People can be exposed to various environmental factors that impact their vision. Using VR, these environmental factors can be simulated, and the impact of these environmental factors on different people can be evaluated using a VR headset equipped with eye-tracking sensors and eye-tracking cameras. Through this assessment, it is possible to identify deficiencies that indicate that a patient is vulnerable to specific conditions. For example, using VR to expose a patient to various environmental factors like smoke and fog can expose ocular conditions such as corneal opacities or Stargardt disease. Similarly, using VR to simulate glare and outdoor lighting can be used to identify cataracts in patients.

A VR system for simulating different environmental conditions and assessing the impact of those environmental conditions on a patient's vision can include a VR headset in electronic communication with a computing device. The VR headset is worn by the patient and can include screens, eye-tracking sensors, and eye-tracking cameras. The screens are configured to display a virtual, three-dimensional environment and different visual tasks. The eye-tracking sensors and the eye-tracking cameras are configured to collect information about the patient's eyes while the patient undergoes the assessment. Specifically, the eye-tracking sensors and the eye-tracking cameras are configured to monitor the patient's gaze direction, fixation duration, and visual acuity. Optionally, the eye-tracking sensors and cameras are infrared sensors and cameras.

Optionally, a handheld device (e.g., a controller, a VR handset, or a glove) is in electronic communication with the VR headset and the computing device. The patient can use the handheld device to interact with the objects in the virtual environment. For example, the patient can also use the handheld device to catch or shoot objects in the virtual environment.

The computing device of the VR system is configured to cause a virtual environment to be displayed on the screens of the VR headset. The computing device can also be configured to cause visual tasks to be displayed in the virtual environment. While the patient completes the visual task, the computing device displays one or more environmental factors (glare, fog, smoke, wind, etc.) over the visual task to obstruct the patient's view of the visual task. The computing device can also adjust the nature or intensity of the environmental factor to test different aspects of the patient's vision and change the difficulty of the visual task.

The computing device can also be configured to receive eye data collected by the VR headset. Optionally, the computing device can include an algorithm that processes the eye data. The algorithm can process the eye data in real-time as the patient completes the visual task.

In some embodiments, the computing device processes the eye data to analyze the amount of time taken by the patient to react to or complete the visual task. In other embodiments, the computing device processes the eye data to evaluate the accuracy of the patient's eye movements. Optionally, the computing device processes the eye data to evaluate the stability of the patient's eye movements.

In some embodiments, the computing device generates a report summarizing the patient's performance (categorized by different environmental factors), diagnosing eye movement disorders, listing treatment and evaluation recommendations, and predicting future eye movement disorders. Optionally, the report highlights eye muscles that the patient may focus on during future training sessions based on the patient's performance in the previous training sessions. The report can also recommend specific training exercises that focus on the recommended muscles. This report can be accessed by a physician, the patient, or the patient's caretaker at a user interface in the computing device.

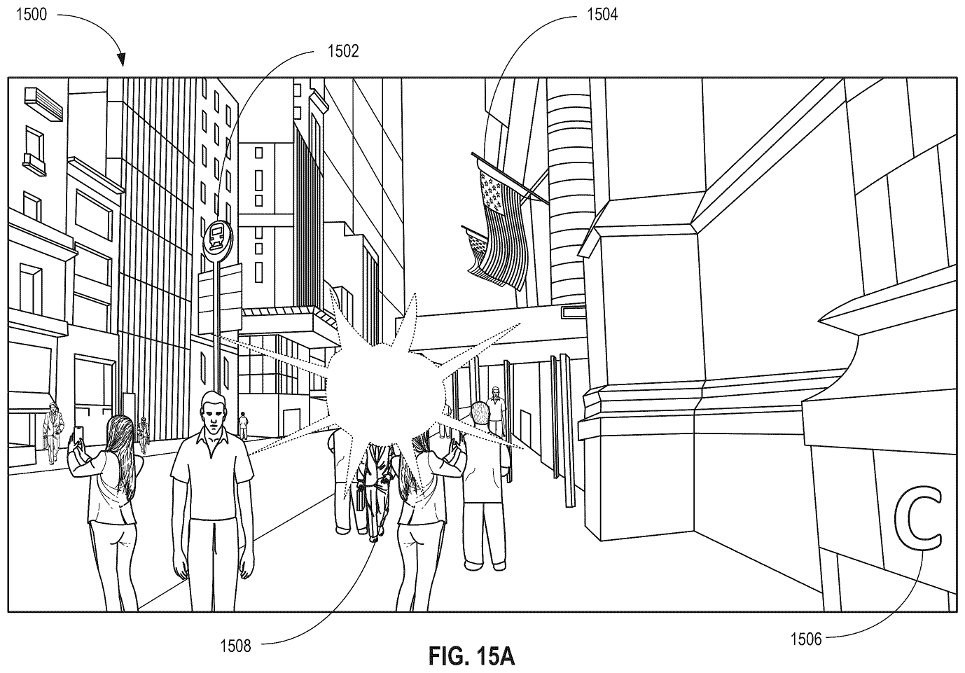

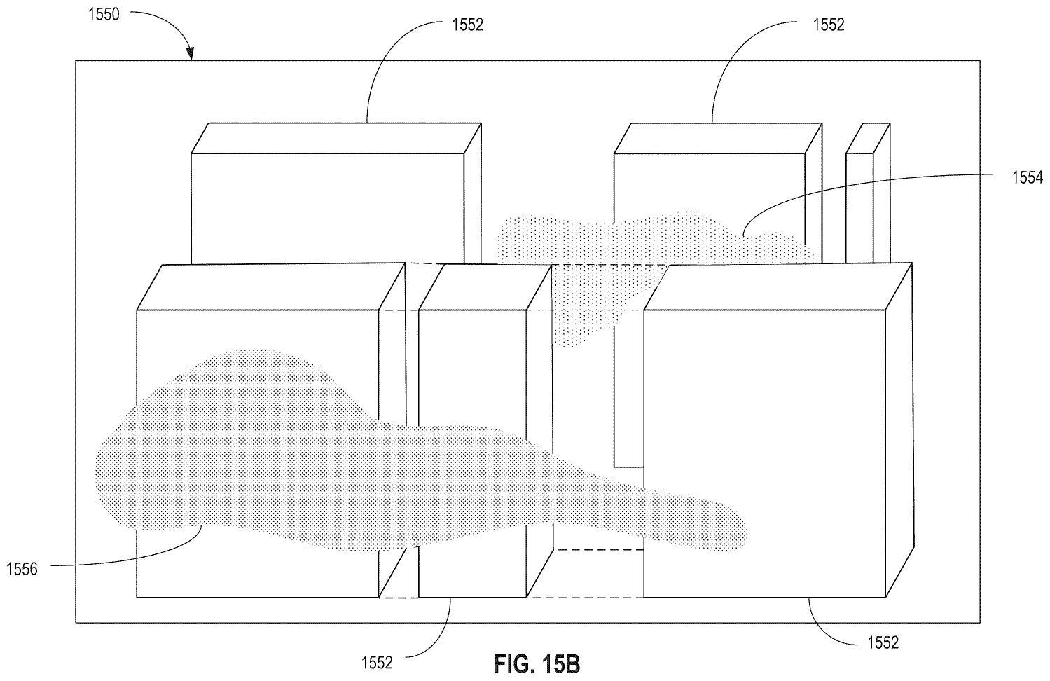

To begin the assessment, the patient can don the VR headset. The computing device causes a virtual environment with different environmental factors to be displayed on the screens of the VR headset and causes a visual task to be displayed in the virtual environment. The different environmental factors can include, at least, glare, fog, smoke, and wind. Glare can be simulated in the virtual environment by increasing the brightness of the virtual environment at a target point that serves as the source of the glare and decreasing the contrast in the areas immediately surrounding the target point. Additionally, at the target point, the glare will obscure the patient's view of the virtual environment. This is demonstrated in FIG. 15A.

FIG. 15A illustrates a virtual environment that is partially obscured by glare, in accordance with some embodiments. As shown, the virtual environment 1500 is a busy city street with various objects, people, and buildings. This virtual environment is very similar to the virtual environment 1350 shown in FIG. 13B. However, the virtual environment 1500 can include a glare 1508 (e.g., from the sun or from the headlights of a vehicle) that obscures some of the people and the buildings in the virtual environment. Because of the decreased contrast and the obscurities, a patient is likely to take longer to complete visual tasks (such as targeting objects or navigating through the crowd) in this environment. This is due to the fact that the patient has to take extra time to see through the different layers of light caused by the glare and to account for the aspects of the virtual environment that the glare is blocking out.

Fog can be simulated in the virtual environment by decreasing the contrast and increasing the brightness across the entirety of the virtual environment. Fog can also be simulated by decreasing the contrast and increasing the brightness across a portion of the virtual environment (e.g., when simulating a cloud of fog), which is depicted in FIG. 15B and described in greater detail below. Optionally, fog can be simulated by displaying white particles in front of the virtual environment to further simulate the manner in which fog can obscure objects in an environment.

Smoke can be simulated in the virtual environment by decreasing both the contrast and the brightness across the entirety or a portion of the virtual environment. FIG. 15B shows a virtual environment that is partially obscured by smoke and is described in greater detail below. Optionally, smoke can be simulated by displaying black particles in front of the virtual environment to simulate the manner in which smoke can obscure objects in an environment. Traditional optical evaluation tools cannot simulate smoke because traditional eyecare tools heavily rely on projectors that shine light into patient's eyes. This means that traditional eyecare tools cannot simulate an environmental factor that is as dark as smoke.

FIG. 15B illustrates a virtual environment that is partially obscured by fog and smoke, in accordance with some embodiments. The virtual environment 1550 in FIG. 15B is an obstacle course and can include two rows of obstacles 1552. As shown, the second row of obstacles 1552 is partially obscured by a cloud of fog 1554. Similarly, the first row of obstacles 1552 is partially obscured by a cloud of smoke 1556. These environmental factors force the patient to see through the fog 1554 or smoke 1556 and/or to guess what the fog 1554 or smoke 1556 is obscuring in order to navigate the obstacle course. Optionally, either the fog 1554 or the smoke 1556 is spread throughout the entirety of the virtual environment. In some embodiments, the intensity of the fog 1554 or smoke 1556 can be adjusted to test different aspects of the patient's visual health or to adapt the vision test to the patient's capabilities.

Another environmental factor that can be displayed in the virtual environment is wind. Wind can be simulated by displaying particles and/or objects moving throughout the virtual environment and across the visual task. For example, the wind can blow through leaves in trees, move the hair and clothing of virtual people in the virtual environment, or even move particles of dirt or dust across the patient's visual field. In some embodiments, the optotype can be depicted as light or flimsy and move when the wind blows. In real life, wind increases the rate at which a patient's tear film evaporates and, thus, decreases the patient's vision. This can be simulated in VR by adding an overall blurring effect to the virtual environment to make the patient feel as though she has a decreased visual acuity.

Various visual tasks can be displayed in the virtual environment with the environmental factors. For example, the patient can be prompted to identify or locate objects and/or optotypes in the virtual environment (e.g., the bus stop 1502, the flag 1504, or the letter “C” 1506 in virtual environment 1500 of FIG. 15A). The patient can also be prompted to navigate through a virtual environment (e.g., navigating through the crows of people in the virtual environment 1500 of FIG. 15A or navigating through the obstacle course in the virtual environment 1550 of FIG. 15B). The patient can also be prompted to track moving objects or optotypes in a virtual environment.

While the patient completes the visual task, eye-tracking sensors and the eye-tracking cameras monitor the patient's eyes and collect input. This can include monitoring the patient's gaze direction, fixation duration, and visual acuity. Monitoring the patient's gaze direction and visual acuity is valuable with respect to ensuring that the patient is correctly focusing on the target object or optotype instead of the environmental factor. For example, a patient will have worse vision if she is focusing on the particulate matter of the fog, wind, or smoke instead of focusing on the optotype. Moreover, monitoring the patient's fixation duration is valuable for determining an amount of time required by the patient to process the different environmental factors. This is particularly valuable for patients who must frequently drive through difficult weather (e.g., people who live in areas with a lot of fog, rain, or snow) or for people who need to accurately track targets in environments with a lot of moving elements (e.g., army soldiers or firefighters).

In some embodiments, the eye-tracking sensors and the eye-tracking cameras monitor the patient's eyes and collect the input in real-time while the patient completes the visual task.

### Evaluating the Effectiveness of Eye Exercises

Many patients partake in eye exercises to improve their vision and visual health. On average, patients must participate in 10-20 eye training sessions to see significant improvements. Typically, after approximately 20 eye training sessions, patients will see a 50% improvement in their vision and/or visual health. By conducting eye training sessions in VR and applying the methods described herein, patient's can see at least an 80% improvement in their vision and/or visual health after approximately 20 eye training sessions.

A VR system for evaluating the effectiveness of eye exercises can include a VR headset having screens, eye-tracking sensors, and eye-tracking cameras. The eye-tracking sensors and cameras are configured to monitor the patient's eyes while she completes the assessments and the eye exercises. In particular, the eye-tracking sensors and eye-tracking cameras are configured to monitor the patient's eye movements, focus adjustments, and visual acuity. In some embodiments, the eye-tracking sensors and eye-tracking cameras monitor the patient's eyes in real-time while the patient completes the assessments and the eye exercises. Optionally, the eye-tracking sensors and cameras are infrared sensors and cameras. Optionally, there is at least one eye-tracking camera pointed at each pupil of the patient when the patient wears the VR headset.

In some embodiments, a handheld device (e.g., a controller or a VR handset) is in electronic communication with the VR headset and the computing device. The patient can use the handheld device to complete the visual assessments and the eye exercises administered on the screens of the VR headset. For example, the patient can press a button to confirm that she is perceiving an object on the screen, use a controller to type in or select a brief description of the object, or even click on the object in the virtual environment.

The VR system further can include a computing device that is configured to conduct the initial and final assessments as well as a series of eye exercises that the patient must partake in as part of her vision raining. This can involve displaying virtual objects and virtual tasks in a virtual environment, as described with respect to other embodiments disclosed in this application.

The computing device can also be configured to process the eye data collected by the eye-tracking sensors and the eye-tracking cameras to evaluate how effectively the eye exercises are improving the patient's vision and/or visual health. In some embodiments, the computing device can comprise an algorithm that is configured to process the eye data to evaluate the patient's visual acuity. In other embodiments, the computing device can comprise an algorithm that is configured to process the eye data to evaluate the patient's eye coordination. Optionally, the computing device can comprise an algorithm that is configured to process the eye data to evaluate the patient's focusing ability. Optionally, the algorithm processes the eye data in real-time as the patient completes the assessments and the eye exercises.

The VR method for evaluating the effectiveness of a patient's eye exercises can begin after the patient dons the VR headset. An initial vision assessment will be administered on the screens of the VR headset. The visual tasks that make up the initial vision assessment are determined, in part, on the aspects of visual health that the patient is targeting with her eye exercises. Generally, visual tasks will evaluate elements such as the patient's eye alignment, focusing ability, visual acuity, eye coordination, and other elements that are described in this disclosure. While the patient completes the initial vision assessment, the eye-tracking sensors and the eye-tracking cameras monitor the patient's eyes to gather information about the patient's responses to the initial vision assessment. This is described above in greater detail.

Next, various eye exercises are conducted on the screens of the VR headset in virtual environments generated by the computing device. These eye exercises can include requiring the patient to focus on an optotype displayed at different depths in the virtual environment (e.g., as previously described with respect to FIG. 13A), requiring the patient to track moving objects and assessing whether the patient's eyes remain aligned throughout tracking, requiring a patient to match or identify optotypes in an array of optotypes (e.g., as previously described with respect to FIGS. 14A-14B-2), or showing the patient two objects and prompting the patient to fuse the objects into a single object in order to evaluate the patient's eye coordination. Other eye exercises are also possible.

After the patient completes the eye exercises, a final vision assessment is conducted in the virtual environment. Optionally, the final vision assessment is identical to the initial vision assessment.

As with the initial vision assessment, the VR headset monitors the patient's eyes during the final vision assessment. The computing device, having the data collected by the VR headset during both the initial and final vision assessments, compares the patient's responses and eye data from the initial vision assessment to the responses and eye data from the final vision assessment to evaluate changes in the patient's vision (i.e., the effect of the eye exercises on the patient's vision and/or visual health). Some examples of this comparison can include comparing the differences in the patient's eye movements, the patient's focus adjustments, and the patient's visual acuity.

In some embodiments, the initial and final vision assessments are conducted before and after every one of the patient's eye training sessions in order to consistently monitor improvement in the patient's vision and/or visual health over time.

### Identifying Visual Stressors in Office Environments and Recommending Ergonomic Adjustments

In any workplace, ergonomics are a crucial consideration to maintaining the health and wellness of employees by reducing musculoskeletal complications and increasing productivity. VR is a valuable tool in the ergonomics space because by simulating workplaces in VR allows a user to quickly experience and test out different furniture and equipment arrangements, which means that the user can quickly and inexpensively determine the parameters of an optimal workplace.

A VR system for identifying visual stressors in the workplace and recommending ergonomic adjustments can include screens, eye-tracking sensors, eye-tracking cameras, and motion-tracking sensors. The screens are configured to display a virtual environment with different pieces of furniture and equipment, different lighting conditions, and other objects to help simulate various workplaces.

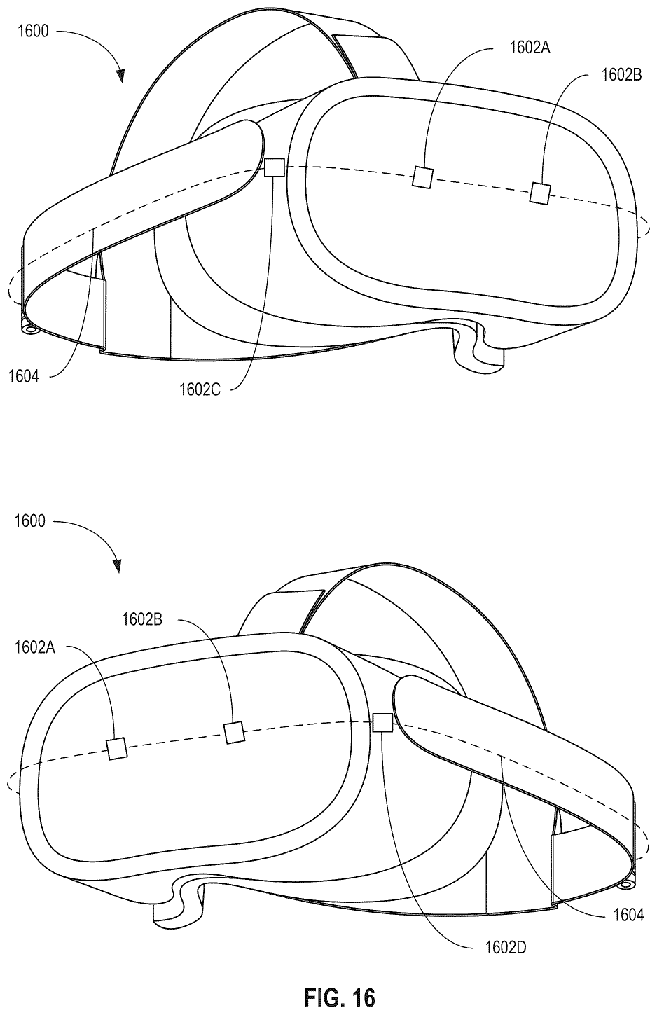

The motion-tracking sensors are configured to track physical movements and a spatial orientation of the user within the virtual environment. The motion-tracking sensors are further configured to track a three-dimensional movement of the head of the user to identify the translation, pitch, yaw, and roll of the user's head as she moves around the virtual environment, tests out different furniture arrangements, etc. In some embodiments, the motion-tracking sensors are configured to track physical movements, spatial orientation, and three-dimensional movement in real-time and communicate the corresponding data to the computing device in real-time. Optionally, the motion-tracking sensors are configured to track a speed at which the user moves her head in different directions. Optionally, the motion-tracking sensors comprise accelerometers and/or gyroscopes. The positions of the motion-tracking sensors on the VR headset are depicted in FIG. 16.

FIG. 16 illustrates a placement of motion-tracking sensors on a VR headset, in accordance with some embodiments. FIG. 16 shows two perspective views of a VR headset 1600 with motion-tracking sensors 1602 (1602A, 1602B, 1602C, and 1602D). Each VR headset 1600 has at least four motion-tracking sensors 1602, with at least two motion-tracking sensors 1602A, 1602B being placed on a front portion of the VR headset 1600, at least one motion-tracking sensor 1602C being placed on a right portion of the VR headset 1600, and at least one motion-tracking sensor 1602D being placed on a left portion of the VR headset 1600. The front portion is the portion of the VR headset 1600 that would be in front of the user's eyes, the right portion would be near the user's right ear, and the left portion would be near the user's left ear. In some embodiments, the motion-tracking sensors 1602 are on the outer exterior of the VR headset 1600 (on the surface of the VR headset 1600 that is away from the user's face), on the inner exterior of the VR headset 1600 (on the surface of the VR headset 1600 that is adjacent to or in contact with the user's face), or on the interior of the VR headset 1600. In some embodiments, the placement of the motion-tracking sensors 1602 is asymmetrical around the user's head.

In some embodiments, the motion-tracking sensors 1602 are positioned along a transverse plane of the VR headset 1600. The transverse plane can align with the eyeline of the user after or when the user dons the VR headset 1600 and is indicated by dashed line 1604 in FIG. 16.

In some embodiments, one or more handheld devices (e.g., a controller or a VR handset) are in electronic communication with the VR headset and the computing device. The user can use the handheld device to interact with the virtual environment displayed on the screens of the VR headset and to otherwise respond to the evaluation. For example, the user can press a button or click on the object to confirm that she has located the object or select the clearing she wants to go through to avoid obstacles.

The eye-tracking sensors and eye-tracking cameras are configured to monitor the user's eye movements, fixation patterns, and blink rates. Monitoring the user's eye movements is valuable because it can be useful to know where the user's natural gaze is. For example, when identifying visual and posture-related stressors for the user in an office setting, the VR headset observes that the user's natural gaze is slightly to the left, the computing device can use that information to recommend that the user place her monitor slightly left of center relative to her desk chair to minimize the amount of eye, neck, or shoulder strain she experiences while working. Similarly, where an office setting has multiple monitors or screens, the computing device can analyze the user's eye movements and fixation patterns to determine whether her eyes are traveling between the different monitors and screens in an efficient manner, which is helpful where, for example, the user gets headaches from looking at her different monitors and screens. Moreover, monitoring the user's blink rate is very valuable for determining whether the user is excessively moving her eyes around her workspace because an increased blink rate correlates with failed attempts to focus on objects, dryness of eyes, and eye strain.

Optionally, the eye-tracking sensors and cameras are infrared sensors and cameras. Optionally, there is at least one eye-tracking camera pointed at each pupil of the patient when the patient wears the VR headset.

The computing device is configured to cause a virtual environment to be displayed on the screens of the VR headset. In this embodiment, the virtual environment is a simulation of the user's workplace. Optionally, the virtual environment is a simulation of the worker's office space. The computing device can also be configured to cause different features in the virtual environment to be changed. For example, the computing device can exchange and rearrange different pieces of furniture and equipment in the virtual environment to maximize the user's comfort and productivity. The computing device can also suggest different orientations of mirrors, monitors, and other reflective items to minimize the amount of glare that a user sees while working.

The computing device can also be configured to process the information collected by the motion-tracking sensors, the eye-tracking sensors, and the eye-tracking cameras. In some embodiments, the computing device can comprise an algorithm that processes this information. Optionally, the algorithm processes this information in real-time. The algorithm can process this information to evaluate the user's visual fatigue, the amount of eye strain that the user is experiencing while working or otherwise engaging with the virtual workplace, and the user's posture.

In some embodiments, the computing device is configured to generate a report summarizing the information collected by the motion-tracking sensors, the eye-tracking sensors, and the eye-tracking cameras as well as the analysis conducted by the computing device by processing this information. Optionally, the computing device can include a user interface at which the report can be accessed.

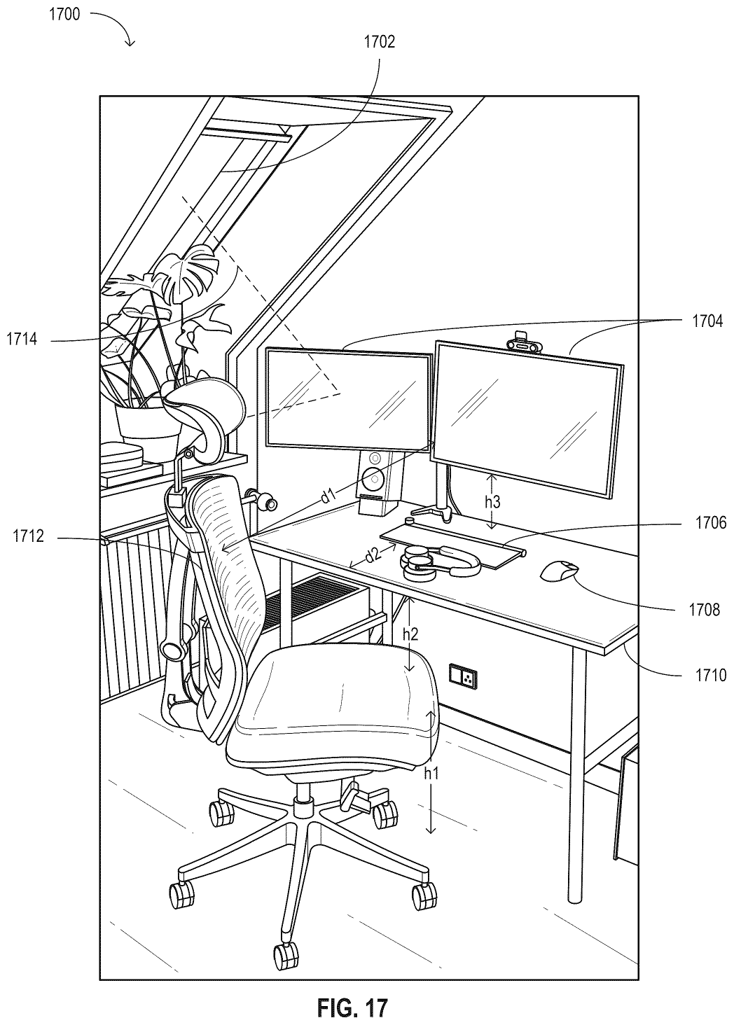

The VR method for identifying and correcting stressors in a workplace can begin after the user dons the VR headset. The computing device causes a virtual workplace to be displayed on the screens of the VR headset. At the beginning of this process, the virtual workplace can be an empty room. Throughout the process, the user will try out different furniture pieces and arrangements until the virtual workplace is a fully functioning, ergonomically optimized workplace such as the one shown in FIG. 17, which is described in greater detail below.

FIG. 17 illustrates a virtual office space that can be furnished and arranged in VR for optimized productivity and comfort, in accordance with some embodiments. The virtual environment 1700 is a virtual home office. Other workspaces (factories, open/shared office spaces, classrooms, etc.) can also be displayed in VR and adjusted using this method. This virtual home office 1700 is lit by a window 1702 and can include computer monitors 1704, a keyboard 1706, a mouse 1706 on a desk 1710 next to an office chair 1712. Each of these features can be individually changed using the VR system in order to optimize the user's comfort and productivity in this workspace. For example, as currently positioned, the window 1702 shines a light 1714 onto the computer monitors 1704, and the computer monitors 1704 reflect the light 1714 into the user's eyes when the user is sitting in the office chair 1712. Using VR, the user can change the height or angle of her monitors 1704, the position of her desk 1710, the height of her chair 1712, etc. to avoid having light 1714 reflected into her eyes while she works. This can also be applicable to light from other sources in the room such as desk lamps or overhead lighting.

Other features that can be modified for the user's comfort and productivity can include the distance d1 between the chair 1712 and the monitors 1704 and the distance d2 between the keyboard 1706 and the edge of the desk 1710. Additionally, the height h1 of the chair 1712 from the ground (e.g., to improve the user's posture by ensuring that the user's shoulders are the correct distance from the desk and that the user's feet touch the ground), the distance h2 between the seat of the chair 1712 and the desk 1710, and the height h3 of the monitors 1704 above the desk 1710 can also be changed. These adjustments can help the user maintain good posture while she is working and reduce the amount of strain her eyes experience while looking at her monitors. Optionally, the user can change the size of different features in her virtual office such as the monitors 1704 and the desk 1710.

In some embodiments, the brightness of the lights in the virtual office space can also be changed. Where the light source is a window, the user can try out different blinds, shades, or curtains in the virtual environment. Where the light source is a lamp or overhead light, the user can adjust the brightness by trying different types of lightbulbs and different placements for the lamps or overhead lights.

Through the VR method, the user can incrementally change features in the virtual environment. Upon changing a first feature or a first set of features, the computing device displays a visual task in the virtual environment. The visual task can include tasks such as having the user read text on virtual computer screens, filling out paperwork on a virtual desk, changing her gaze between different virtual computer screens, etc. Optionally, the user can complete any of the other visual tasks described in this application such as reading an array of optotypes, tracking moving objects, etc.

While the user completes these visual tasks, the VR headset monitors the user's eyes and posture, and the computing device processes this information to identify sources of visual stress, identify sources of physical discomfort, and recommend further adjustments to the virtual office space. These steps are described in greater detail above. The process of changing features of the virtual workspace, conducting visual tasks to test out the ergonomics of the changed features, and recommending additional changes is repeated until the user's virtual office space is optimized for comfort, visual acuity, and productivity.

In some embodiments, the VR system prompts the user to provide her input regarding her visual acuity and ergonomic comfort in the virtual environment. This subjective input can be received via a handheld device or in the form of verbal input from the user received via microphones on the VR headset. The computing device can process this subjective input and use it to suggest changes to various features of the virtual office space. Optionally, this prompt occurs incrementally throughout the implementation of this VR method. For example, the user might be prompted every five minutes or even every 30-40 seconds.

### Simulating UV Exposure and Measuring the Effects of UV Exposure

Exposure to UV light from the sun is an inevitable part of life, but it can also have detrimental effects on a person's visual health. For example, exposure to UV light can lead to cataracts or retinal scars. Both of which can significantly degrade a person's vision. For these reasons, people need to learn how much UV light they are exposed to on a regular basis and how to protect themselves from this exposure. It can also be important to measure the effects of UV exposure on a person's eyes to treat ocular conditions like cataracts and retinal scars in their early stages.

A VR system for simulating real-life scenarios in which a user might be exposed to UV light and for measuring the effects of UV light exposure on the user's eyes can include a VR headset in electronic communication with a computing device. The VR headset can include screens, eye-tracking sensors, and eye-tracking cameras. The screens are configured to display a three-dimensional, virtual environment with various lighting conditions for the user wearing the VR headset.

The eye-tracking sensors and the eye-tracking cameras monitor the user's eyes to collect eye data such as the user's gaze direction, blink rate, and pupillary responses. These factors are indicative of how much UV light exposure the user's eyes receive in different scenarios and also provide information about retinal damage and the formation of cataracts. For example, damage to the retina can lead to a higher blink rate (almost double the average blink rate, in many situations) and pupils that tend to remain dilated. Optionally, the eye-tracking sensors and cameras are infrared sensors and cameras.

The computing device is configured to cause a virtual environment to be displayed on the screens of the VR headset. The computing device can also be configured to simulate UV light in the virtual environment and to cause visual tasks to be displayed in the virtual environment. Optionally, the computing device is configured to display a lecture or lesson for the user that explains the sources of UV light in various environments and the consequences of exposure to the UV light.

The computing device can also be configured to process the eye data collected by the eye-tracking sensors and the eye-tracking cameras to evaluate the degree to which the UV light exposure has impacted the user's visual health. In some embodiments, the computing device can comprise an algorithm that is configured to process the eye data to evaluate the user's blink frequency. In other embodiments, the computing device can comprise an algorithm that is configured to process the eye data to evaluate an extent to which the user squints when exposed to UV light. Optionally, the computing device can comprise an algorithm that is configured to process the eye data to evaluate the pupil constrictions of the user. Optionally, the algorithm processes the eye data in real-time as the patient observes the virtual environment.

In some embodiments, the computing device is configured to generate a report recommending UV protective measures for the user based on how UV light has affected the user's visual health. Optionally, the computing device further can include a user interface at which this report can be accessed and shared.

In some embodiments, a method for simulating UV light exposure is provided to teach the user different scenarios in which she might be exposed to UV light and how to protect her eyes in those scenarios.

For example, the VR lessons might teach the user that she is exposed to UV light from light reflecting from a body of water in addition to direct sunlight. The user can don the VR headset and sees virtual environments with different lighting conditions (e.g., direct sunlight, a cloudy day, reflected light, sunlight reaching indoor spaces, etc.). The VR system accomplishes these various lighting conditions by changing the brightness of the virtual environment and the direction from which the light reaches the user.

Upon displaying these different virtual environments and lighting conditions for the user, the VR system also explains why these different scenarios expose her to UV light and how she can protect herself in each scenario.

Some embodiments of the present disclosure provide a method for measuring the effects of UV light exposure on the user. After the user dons the VR headset, virtual environments with different lighting conditions (as described above) can be displayed on the screens. As previously described, the eye-tracking sensors and the eye-tracking cameras monitor the user's gaze direction, blink rate, and pupillary responses. Optionally, this monitoring occurs continuously or in real-time as the user observes the virtual environment.

The computing device processes this information collected by the VR headset to evaluate the effects of the UV light on the user and to recommend different protective measures such as squinting, wearing hats and sunglasses, or walking in the shade.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A method for assessing focusing ability, the method comprising: displaying a virtual environment on screens of a virtual reality (VR) headset worn by a patient; displaying an object at a first position in the virtual environment; prompting the patient to focus on the object at the first position; monitoring a first eye position and a first pupil size of the patient as the patient focuses on the object in the first position; displaying the object at a second position in the virtual environment, wherein the second position has a different depth in the virtual environment than the first position; prompting the patient to focus on the object at the second position; monitoring a second eye position and a second pupil size of the patient as the patient focuses on the object in the second position to evaluate a transition from the first eye position to the second eye position and from the first pupil size to the second pupil size in response to the object being displayed at the second position; calculating a focus time of the patient, wherein the focus time comprises one or more of (a) a time taken by the patient to transition between the first eye position and the second eye position and (b) a time taken by the patient to transition between the first pupil size and the second pupil size; and comparing the focus time to a database to assess a focusing ability of the patient.
Clause 2. The method of Clause 1, wherein displaying the object at the second position comprises moving the object from the first position to the second position along a path.
Clause 3. The method of any of the preceding Clauses, wherein displaying the object at the second position comprises displaying the object at a position near the patient, and the method further comprises prompting the patient to catch the object when the object reaches the second position.
Clause 4. The method of any of the preceding Clauses, further comprising prompting the patient to shoot the object when the object reaches the second position.
Clause 5. The method of any of the preceding Clauses, wherein displaying the object at the second position comprises removing the object from the first position and the virtual environment and placing the object in the second position.
Clause 6. The method of any of the preceding Clauses, wherein the first position is further from the patient than the second position.
Clause 7. The method of any of the preceding Clauses, wherein the second position is further from the patient than the first position.
Clause 8. The method of any of the preceding Clauses, wherein monitoring the first eye position, the first pupil size, the second eye position, and the second pupil size comprises monitoring in real-time as the patient responds to the virtual environment.
Clause 9. The method of any of the preceding Clauses, wherein calculating a focus time of the patient comprises calculating the focus time in real-time as the patient responds to the virtual environment.
Clause 10. The method of any of the preceding Clauses, further comprising displaying the object at a third position in the virtual environment, wherein the third position is determined by the first and second eye positions and the first and second pupil sizes.
Clause 11. A method for assessing focusing ability, the method comprising: displaying a plurality of objects in a virtual environment on screens of a virtual reality (VR) headset worn by a patient, wherein each object comprises a characteristic and is positioned at a distance from the patient; prompting the patient to focus on a first object of the plurality of objects, wherein the first object has a first characteristic and is positioned at a first distance from the patient; monitoring a first eye position and a first pupil size of the patient as the patient focuses on the first object; prompting the patient to focus on a second object of the plurality of objects, wherein the second object has a second characteristic and is positioned at a second distance from the patient, the second characteristic and the second distance being different from the first characteristic and the first distance; monitoring a second eye position and a second pupil size of the patient as the patient focuses on the second object to evaluate a transition from the first eye position to the second eye position and from the first pupil size to the second pupil size in response to looking at the second object after looking at the first object; calculating a focus time of the patient, wherein the focus time comprises one or more of (a) a time taken by the patient to transition between the first eye position and the second eye position and (b) a time taken by the patient to transition between the first pupil size and the second pupil size; and comparing the focus time to a database to assess a focusing ability of the patient.
Clause 12. The method of Clause 11, wherein displaying the plurality of objects in the virtual environment comprises displaying a first plurality of objects at a first juncture of a maze.
Clause 13. The method of any of Clauses 11 through 12, further comprising prompting the patient to focus on a target object of the first plurality of objects and navigating the patient through the first juncture when the patient focuses on the target object.
Clause 14. The method of any of Clauses 11 through 13, wherein prompting the patient to focus on the first object occurs at a first time and prompting the patient to focus on the second object occurs at a second time, and the method further comprises changing a number of milliseconds between the first time and the second time.
Clause 15. The method of any of Clauses 11 through 14, wherein displaying the plurality of objects comprises displaying the first object or the second object centrally in a patient's field of vision.
Clause 16. The method of any of Clauses 11 through 15, wherein displaying the plurality of objects comprises displaying the first object or the second object is peripherally in a patient's field of vision.
Clause 17. The method of any of Clauses 11 through 16, wherein displaying the plurality of objects comprises displaying the first object centrally in a patient's field of vision and displaying the second object peripherally in a patient's field of vision.
Clause 18. The method of any of Clauses 11 through 17, wherein monitoring the first eye position, the first pupil size, the second eye position, and the second pupil size comprises monitoring in real-time as the patient responds to the prompts.
Clause 19. The method of any of Clauses 11 through 18, wherein calculating a focus time of the patient comprises calculating the focus time in real-time as the patient responds to the prompts.
Clause 20. The method of any of Clauses 11 through 19, further comprising prompting the patient to focus on a third object of the plurality of objects, wherein the third object has a third characteristic and is positioned at a third distance from the patient, the third characteristic and the third distance being determined by the first and second eye positions and the first and second pupil sizes.
Clause 21. The method of any of Clauses 11 through 20, wherein the first characteristic comprises a color of the first object and the second characteristic comprises the color of the second object.
Clause 22. The method of any of Clauses 11 through 21, wherein the first characteristic comprises a size of the first object and the second characteristic comprises the size of the second object.
Clause 23. The method of any of Clauses 11 through 22, wherein the first characteristic comprises a contrast of the first object and the second characteristic comprises the contrast of the second object.
Clause 24. The method of any of Clauses 11 through 23, wherein the first characteristic comprises a brightness of the first object and the second characteristic comprises the brightness of the second object.
Clause 25. The method of any of Clauses 11 through 24, wherein the first characteristic comprises a shape of the first object and the second characteristic comprises the shape of the second object.
Clause 26. The method of any of Clauses 11 through 25, wherein the first distance is further from the patient than the second distance.
Clause 27. The method of any of Clauses 11 through 26, wherein the second distance is further from the patient than the first distance.
Clause 28. A system for assessing focusing ability, the system comprising: a virtual reality (VR) headset worn by a patient, the VR headset comprising screens, one or more eye-tracking sensors, and one or more eye-tracking cameras, wherein the one or more eye-tracking sensors and the one or more eye-tracking cameras are configured to collect focusing data as the patient responds to virtual environments displayed on the screens; and a computing device in electronic communication with the VR headset, the computing device being configured to cause virtual environments to be displayed on the screens, prompt the patient to focus on objects in the virtual environment, and process the focusing data to evaluate the focusing ability of the patient.
Clause 29. The system of Clause 28, wherein the one or more eye-tracking sensors are configured to monitor a pupil size of the patient, and the computing device is configured to process the pupil size to calculate a depth of focus of the patient.
Clause 30. The system of any of Clauses 28 through 29, wherein the one or more eye-tracking cameras are configured to monitor an eye position of the patient, and the computing device is configured to process the eye position to calculate a depth of focus of the patient.
Clause 31. The system of any of Clauses 28 through 30, wherein the one or more eye-tracking cameras comprise infrared cameras.
Clause 32. The system of any of Clauses 28 through 31, wherein the focusing data comprises eye movements of the patient.
Clause 33. The system of any of Clauses 28 through 32, wherein the focusing data comprises focus adjustments of the patient.
Clause 34. The system of any of Clauses 28 through 33, wherein the focusing data comprises reaction times of the patient.
Clause 35. The system of any of Clauses 28 through 34, wherein the computing device comprises an algorithm that processes the focusing data to evaluate a focusing speed of the patient.
Clause 36. The system of any of Clauses 28 through 35, wherein the computing device comprises an algorithm that processes the focusing data to evaluate a focusing accuracy of the patient.
Clause 37. The system of any of Clauses 28 through 36, wherein the computing device comprises an algorithm that processes the focusing data to evaluate a focusing consistency of the patient.
Clause 38. The system of any of Clauses 28 through 37, wherein the computing device comprises an algorithm that processes the focusing data in real-time as the patient focuses on the objects in the virtual environment.
Clause 39. The system of any of Clauses 28 through 38, wherein the computing device comprises an algorithm that processes the focusing data to identify deficiencies in the focusing ability of the patient that are indicative of accommodative dysfunction.
Clause 40. The system of any of Clauses 28 through 39, wherein the computing device comprises an algorithm that processes the focusing data to identify deficiencies in the focusing ability of the patient that are indicative of presbyopia.
Clause 41. The system of any of Clauses 28 through 40, wherein the computing device is configured to cause objects to be displayed in the virtual environments and calculate positions of the objects in the virtual environments.
Clause 42. The system of any of Clauses 28 through 41, wherein the one or more eye-tracking cameras is configured to monitor an eye position of the patient, and the computing device is configured to process the eye position to determine a difference between the eye position and the position of the one or more objects.
Clause 43. The system of any of Clauses 28 through 42, wherein the computing device is configured to calculate an accuracy of the eye position.
Clause 44. The system of any of Clauses 28 through 43, wherein the computing device is configured to prompt the patient to interact with the objects in the virtual environment, and the system further comprises a handheld device in electronic communication with the VR headset and the computing device, wherein the handheld device is configured to receive input from the patient as the patient interacts with the objects.
Clause 45. A virtual reality (VR) headset for assessing focusing ability, the VR headset comprising screens configured to display a virtual environment for a patient wearing the VR headset, one or more eye-tracking sensors, and one or more eye-tracking cameras.
Clause 46. The VR headset of Clause 45, wherein the one or more eye-tracking cameras comprises at least one eye-tracking camera facing each eye of the patient wearing the VR headset.
Clause 47. The VR headset of any of Clauses 45 through 46, wherein the one or more eye-tracking sensors and the one or more eye-tracking cameras are configured to monitor a position of each eye of the patient.
Clause 48. The VR headset of any of Clauses 45 through 47, wherein the one or more eye-tracking sensors and the one or more eye-tracking cameras are configured to monitor a pupil size of each eye of the patient.
Clause 49. A method for training ocular muscles, the method comprising: displaying a virtual environment and an object on screens of a virtual reality (VR) headset worn by a patient, wherein the object is positioned at a first 3D coordinate within the virtual environment; prompting the patient to visually track the object; moving the object to a second 3D coordinate within the virtual environment; monitoring the patient's eyes to collect a first input; moving the object to a third 3D coordinate within the virtual environment; and monitoring the patient's eyes to collect a second input.
Clause 50. The method of Clause 49, wherein displaying the object comprises displaying a circle or a ring.
Clause 51. The method of any of Clauses 49 through 50, wherein displaying the object at the first, second, and third 3D coordinates comprises displaying the object at positions with different x, y, and/or z coordinates, wherein the x coordinate corresponds to an x-axis that is left and right relative to the patient, the y coordinate corresponds to a y-axis that is up and down relative to the patient, and the z coordinate corresponds to a z-axis that is away from and towards the patient.
Clause 52. The method of any of Clauses 49 through 51, wherein moving the object to the second and third 3D coordinates comprises moving the object along one or more of the x-axis, the y-axis, or the z-axis.
Clause 53. The method of any of Clauses 49 through 52, wherein moving the object to the second or third 3D coordinate comprises moving the object along a smooth path.
Clause 54. The method of any of Clauses 49 through 53, wherein moving the object to the second or third 3D coordinate comprises moving the object along a jagged path.
Clause 55. The method of any of Clauses 49 through 54, wherein moving the object to the second or third 3D coordinate comprises moving the object along a randomized path.
Clause 56. The method of any of Clauses 49 through 55, wherein moving the object to the second or third 3D coordinate comprises moving the object along a straight line.
Clause 57. The method of any of Clauses 49 through 56, wherein moving the object to the second or third 3D coordinate comprises moving the object along a path shaped like an alphanumeric character.
Clause 58. The method of any of Clauses 49 through 57, wherein displaying the object at the first 3D coordinate and moving the object to the second and third 3D coordinates comprises displaying the object at the first 3D coordinate at a first time and moving the object to the second and third 3D coordinates at a second time and a third time, respectively, and the method further comprises changing a number of milliseconds between the first time, the second time, and/or the third time.
Clause 59. The method of any of Clauses 49 through 58, wherein monitoring the eyes of the patient comprises continuously monitoring the eyes as the patient tracks the object.
Clause 60. The method of any of Clauses 49 through 59, further comprising providing feedback, wherein the feedback is configured to indicate a degree to which the patient is successfully tracking the object.
Clause 61. The method of any of Clauses 49 through 60, wherein providing the feedback comprises providing haptic feedback.
Clause 62. The method of any of Clauses 49 through 61, wherein providing the feedback comprises providing audio feedback.
Clause 63. The method of any of Clauses 49 through 62, further comprising, when the first and second inputs suggest that the patient is successfully tracking the object, increasing one or more of a field of vision in which the object is displayed or a speed at which the object moves.
Clause 64. The method of any of Clauses 49 through 63, further comprising, when the first and second inputs suggest that the patient is not tracking the object, decreasing one or more of a field of vision in which the object is displayed or a speed at which the object moves.
Clause 65. The method of any of Clauses 49 through 64, further comprising, after prompting the patient to track the object, occluding one of the patient's eyes.
Clause 66. The method of any of Clauses 49 through 65, further comprising changing an orientation of the object as the object is moved to the second and third 3D coordinates.
Clause 67. The method of any of Clauses 49 through 66, further comprising generating a report that highlights eye muscles that the patient should focus on during future training sessions based on one or more of the first or second input.
Clause 68. The method of any of Clauses 49 through 67, further comprising generating a report that recommends training exercises for the patient based on one or more of the first or second input.
Clause 69. A system for training ocular muscles, the system comprising: a virtual reality (VR) headset worn by a patient, the VR headset comprising screens, one or more eye-tracking sensors, one or more eye-tracking cameras, speakers, and vibrating motors, the one or more eye-tracking sensors and cameras being configured to collect eye movement data from the patient; and a computing device in electronic communication with the VR headset, the computing device being configured to cause a virtual environment to be displayed on the screens, to cause an object to move in three dimensions throughout the virtual environment, and to process the eye movement data, wherein the eye movement data is collected by the one or more eye-tracking sensors and cameras while the patient tracks the object throughout the virtual environment.
Clause 70. The system of Clause 69, wherein the one or more eye-tracking cameras comprises an infrared camera.
Clause 71. The system of any of Clauses 69 to 70, wherein the speakers are configured to provide audio feedback to the patient as the patient tracks the object through the virtual environment.
Clause 72. The system of any of Clauses 69 to 71, wherein the vibrating motors are configured to provide haptic feedback to the patient as the patient tracks the object through the virtual environment.
Clause 73. The system of any of Clauses 69 to 72, wherein the computing device comprises an algorithm configured to process the eye movement data in real-time as the patient tracks the object throughout the virtual environment.
Clause 74. The system of any of Clauses 69 to 73, wherein the computing device comprises an algorithm configured to process the eye movement data to evaluate a degree to which the patient is successfully tracking the object.
Clause 75. The system of any of Clauses 69 to 74, wherein the computing device comprises an algorithm configured to adapt a movement of the object based on a degree to which the patient is successfully tracking the object.
Clause 76. The system of any of Clauses 69 to 75, wherein the computing device is further configured to generate a report and comprises a user interface at which the report can be accessed.
Clause 77. A virtual reality (VR) headset for training ocular muscles, the VR headset comprising screens, one or more eye-tracking sensors, one or more eye-tracking cameras, speakers, and vibrating motors, wherein the one or more eye-tracking sensors and cameras are configured to monitor eye movements of the patient while the patient reacts to a virtual environment displayed on the screens.
Clause 78. A method for vision training using feedback-adjusted visual challenges, the method comprising: displaying a virtual environment on screens of a virtual reality (VR) headset worn by a patient; displaying an array of optotypes in the virtual environment, wherein each optotype of the array of optotypes comprises an orientation, a contrast, and/or a color that is distinct from the orientation, the contrast, and/or the color of at least one other optotype of the array of optotypes; prompting the patient to match a first model optotype with a first target optotype in the array of optotypes; monitoring a patient's eyes to collect a first input as the patient attempts to match the first model optotype with the first target optotype; based on the first input, adjusting the orientation, the contrast, and the color of one or more optotype in the array of optotypes; and prompting the patient to match a second model optotype with a second target optotype in the array of optotypes.
Clause 79. The method of Clause 78, wherein displaying the virtual environment comprises displaying a white background, and displaying the array of optotypes comprises displaying the array of optotypes, wherein each optotype is black.
Clause 80. The method of any of Clauses 78 through 79, wherein displaying the array of optotypes comprises displaying the array of optotypes, wherein a size of each optotype in the array of optotypes incrementally decreases throughout the array.
Clause 81. The method of any of Clauses 78 through 80, wherein monitoring the patient's eyes to collect the first input comprises continuously monitoring the patient's eyes.
Clause 82. The method of any of Clauses 78 through 81, wherein monitoring the patient's eyes to collect the first input comprises monitoring eye movements of the patient.
Clause 83. The method of any of Clauses 78 through 82, wherein monitoring the patient's eyes to collect the first input comprises monitoring eye movements and pupillary responses of the patient.
Clause 84. The method of any of Clauses 78 through 83, wherein monitoring the patient's eyes to collect the first input comprises monitoring a time taken by the patient to match the first model optotype with the first target optotype.
Clause 85. The method of any of Clauses 78 through 84, further comprising: monitoring the patient's eyes to collect a second input as the patient attempts to match the second model optotype with the second target optotype; and when the first and second inputs suggest that the patient is successfully matching the first and second model optotypes with the first and second target optotypes, respectively, decreasing the contrast of each optotype in the array of optotypes.
Clause 86. The method of any of Clauses 78 through 85, further comprising: monitoring the patient's eyes to collect a second input as the patient attempts to match the second model optotype with the second target optotype; and when the first and second inputs suggest that the patient is not matching the first and second model optotypes with the first and second target optotypes, respectively, increasing the contrast of each optotype in the array of optotypes.
Clause 87. The method of any of Clauses 78 through 86, further comprising: monitoring the patient's eyes to collect a second input as the patient attempts to match the second model optotype with the second target optotype; and processing one or more of the first and second inputs to analyze an eye movement accuracy of the patient.
Clause 88. The method of any of Clauses 78 through 87, further comprising: monitoring the patient's eyes to collect a second input as the patient attempts to match the second model optotype with the second target optotype; and processing one or more of the first and second inputs to analyze an eye movement speed of the patient.
Clause 89. The method of any of Clauses 78 through 88, further comprising: monitoring the patient's eyes to collect a second input as the patient attempts to match the second model optotype with the second target optotype; and processing one or more of the first and second inputs to analyze an eye movement stability of the patient.
Clause 90. A method for vision training using feedback-adjusted visual challenges, the method comprising: displaying a virtual environment on screens of a virtual reality (VR) headset worn by a patient; displaying a visual task at a first position in the virtual environment; prompting the patient to complete the visual task while the visual task is at the first position; monitoring a patient's eyes to collect a first input as the patient attempts to complete the visual task; based on the first input, moving the visual task from the first position to a second position in the virtual environment; and prompting the patient to complete the visual task while the visual task is at the second position.
Clause 91. The method of Clause 90, wherein displaying the visual task comprises displaying an array of optotypes.
Clause 92. The method of any of Clauses 90 through 91, further comprising changing a type or difficulty of the visual task when moving the visual task from the first position to the second position.
Clause 93. The method of any of Clauses 90 through 93, wherein moving the visual task from the first position to the second position comprises changing a position of the visual task along one or more of an x-axis, a y-axis, or a z-axis, wherein the x-axis extends left and right relative to the patient, the y-axis extends up and down relative to the patient, and the z-axis extends away from and towards the patient.
Clause 94. The method of any of Clauses 90 through 93, wherein moving the visual task from the first position to the second position comprises removing the visual task from the virtual environment at the first position and displaying the visual task in the virtual environment at the second position.
Clause 95. The method of any of Clauses 90 through 94, wherein moving the visual task from the first position to the second position comprises moving the visual task along a path from the first position to the second position.
Clause 96. The method of any of Clauses 90 through 95, wherein moving the visual task from the first position to the second position comprises placing the visual task at an unpredictable position in the virtual environment.
Clause 97. The method of any of Clauses 90 through 96, wherein displaying the visual task at the first position occurs at a first time, wherein moving the visual task from the first position to the second position occurs at a second time, and the method further comprises changing a number of milliseconds between the first time and the second time.
Clause 98. The method of any of Clauses 90 through 97, wherein monitoring the patient's eyes to collect the first input comprises continuously monitoring the patient's eyes as the patient attempts to complete the visual task.
Clause 99. The method of any of Clauses 90 through 98, wherein monitoring the patient's eyes to collect the first input comprises monitoring eye movements of the patient.
Clause 100. The method of any of Clauses 90 through 99, wherein monitoring the patient's eyes to collect the first input comprises monitoring eye movements and pupillary responses of the patient.
Clause 101. The method of any of Clauses 90 through 100, wherein monitoring the patient's eyes to collect the first input comprises monitoring a time taken by the patient to complete the visual task when the visual task is at the first position.
Clause 102. The method of any of Clauses 90 through 101, further comprising: monitoring the patient's eyes to collect a second input as the patient attempts to complete the visual task when the visual task is at the second position; and when the first and second inputs suggest that the patient is successfully completing the visual task, increasing a difficulty of the visual task.
Clause 103. The method of any of Clauses 90 through 102, further comprising: monitoring the patient's eyes to collect a second input as the patient attempts to complete the visual task when the visual task is at the second position; and when the first and second inputs suggest that the patient is improperly completing the visual task, decreasing the difficulty of the visual task.
Clause 104. The method of any of Clauses 90 through 103, further comprising processing the first input to analyze an eye movement accuracy of the patient.
Clause 105. The method of any of Clauses 90 through 104, further comprising processing the first input to analyze an eye movement speed of the patient.
Clause 106. The method of any of Clauses 90 through 105, further comprising processing the first input to analyze an eye movement stability of the patient.
Clause 107. The method of any of Clauses 90 through 106, further comprising generating a report that highlights areas of a patient's visual health that require further training.
Clause 108. A system for vision training using feedback-adjusted visual challenges, the system comprising: a virtual reality (VR) headset worn by a patient, the VR headset comprising screens, one or more eye-tracking sensors, and one or more eye-tracking cameras, the one or more eye-tracking sensors and cameras being configured to collect eye data; and a computing device in electronic communication with the VR headset, the computing device being configured to cause a virtual environment to be displayed on the screens, to cause a visual task to be displayed in the virtual environment, and to process the eye data to adjust the visual task, wherein the eye data comprises eye movements and fixation points of the patient while the patient completes the visual task.
Clause 109. The system of Clause 108, further comprising a handheld device in electronic communication with the VR headset and the computing device, wherein the handheld device is configured to receive input from the patient as the patient completes the visual task.
Clause 110. The system of any of Clauses 108 through 109, wherein the one or more eye-tracking cameras comprises an infrared camera.
Clause 111. The system of any of Clauses 108 through 110, wherein the computing device is configured to process the eye data and calculate an amount of time taken by the patient to complete the visual task.
Clause 112. The system of any of Clauses 108 through 111, wherein the computing device is configured to process the eye data and calculate the amount of time in real-time.
Clause 113. The system of any of Clauses 108 through 112, wherein the computing device comprises an algorithm configured to process the eye data to analyze an eye movement accuracy of the patient.
Clause 114. The system of any of Clauses 108 through 113, wherein the computing device comprises an algorithm configured to process the eye data to analyze an eye movement speed of the patient.
Clause 115. The system of any of Clauses 108 through 114, wherein the computing device comprises an algorithm configured to process the eye data to analyze an eye movement stability of the patient.
Clause 116. The system of any of Clauses 108 through 115, wherein the algorithm is configured to process the eye data in real-time.
Clause 117. A method for assessing the impact of environmental factors on vision, the method comprising: displaying a virtual environment on screens of a virtual reality (VR) headset worn by a patient; displaying a visual task in the virtual environment with an environmental factor; prompting the patient to complete the visual task while the virtual environment includes the environmental factor; and monitoring a patient's eyes to collect an input as the patient attempts the visual task.
Clause 118. The method of Clause 117, wherein displaying the visual task in the virtual environment with the environmental factor comprises displaying the visual task in the virtual environment with glare.
Clause 119. The method of any of Clauses 117 through 118, wherein displaying the visual task in the virtual environment with glare comprises increasing brightness of the virtual environment and the visual task at one or more target points, decreasing contrast of the virtual environment and the visual task in areas surrounding the one or more target points, and obscuring the virtual environment and the visual task at the one or more target points.
Clause 120. The method of any of Clauses 117 through 119, wherein displaying the visual task in the virtual environment with the environmental factor comprises displaying the visual task in the virtual environment with fog.
Clause 121. The method of any of Clauses 117 through 120, wherein displaying the visual task in the virtual environment with fog comprises decreasing contrast and increasing brightness across the virtual environment and the visual task.
Clause 122. The method of any of Clauses 117 through 121, wherein displaying the visual task in the virtual environment with the environmental factor comprises displaying the visual task in the virtual environment with smoke.
Clause 123. The method of any of Clauses 117 through 122, wherein displaying the visual task in the virtual environment with smoke comprises decreasing contrast and brightness across virtual environment and the visual task and displaying dark particles across the virtual environment and the virtual task.
Clause 124. The method of any of Clauses 117 through 123, wherein displaying the visual task in the virtual environment with the environmental factor comprises displaying the visual task in the virtual environment with wind.
Clause 125. The method of any of Clauses 117 through 124, wherein displaying the visual task in the virtual environment with wind comprises displaying particles and/or objects moving throughout the virtual environment and the visual task.
Clause 126. The method of any of Clauses 117 through 125, wherein displaying a visual task comprises displaying one or more optotypes in the virtual environment and prompting the patient to identify the one or more optotypes.
Clause 127. The method of any of Clauses 117 through 126, wherein displaying a visual task comprises displaying one or more optotypes in the virtual environment and prompting the patient to locate the one or more optotypes.
Clause 128. The method of any of Clauses 117 through 127, wherein displaying a visual task comprises displaying a moving optotype throughout the virtual environment and prompting the patient to track the moving optotype.
Clause 129. The method of any of Clauses 117 through 128, wherein monitoring the patient's eyes to collect the input comprises monitoring the patient's eyes in real-time as the patient attempts the visual task.
Clause 130. The method of any of Clauses 117 through 129, wherein monitoring the patient's eyes to collect the input comprises monitoring a gaze direction of the patient.
Clause 131. The method of any of Clauses 117 through 130, wherein monitoring the patient's eyes to collect the input comprises monitoring a fixation duration of the patient.
Clause 132. The method of any of Clauses 117 through 131, wherein monitoring the patient's eyes to collect the input comprises monitoring a visual acuity of the patient.
Clause 133. The method of any of Clauses 117 through 132, further comprising displaying the visual task in the virtual environment with at least two environmental factors.
Clause 134. The method of any of Clauses 117 through 133, further comprising adjusting a type or severity of the environmental factor.
Clause 135. The method of any of Clauses 117 through 134, further comprising: displaying the visual task in virtual environment with a different environmental factor; prompting the patient to complete the visual task while the virtual environment includes the different environmental factor; and monitoring the patient's eyes to collect another input as the patient attempts the visual task.
Clause 136. The method of any of Clauses 117 through 135, wherein a type and severity of the different environmental factor is determined by analyzing the input.
Clause 137. The method of any of Clauses 117 through 136, further comprising processing the input to evaluate a reaction time of the patient.
Clause 138. The method of any of Clauses 117 through 137, further comprising processing the input to evaluate an eye movement accuracy of the patient.
Clause 139. The method of any of Clauses 117 through 138, further comprising processing the input to evaluate an eye movement stability of the patient.
Clause 140. The method of any of Clauses 117 through 139, further comprising comparing the input to a database to assess the impact of the environmental factor on a patient's vision.
Clause 141. The method of any of Clauses 117 through 140, further comprising generating a report that summarizes patient's performance, categorized by different environmental factors, and identifying deficiencies.
Clause 142. A system for assessing the impact of environmental factors on vision, the system comprising: a virtual reality (VR) headset worn by a patient, the VR headset comprising screens, one or more eye-tracking sensors, and one or more eye-tracking cameras, the one or more eye-tracking sensors and cameras being configured to collect eye data; and a computing device in electronic communication with the VR headset, the computing device being configured to cause a virtual environment with one or more environmental factors to be displayed on the screens, to cause a visual task to be displayed in the virtual environment, to adjust the one or more environmental factors, and to process the eye data, wherein the eye data comprises gaze direction, fixation duration, and visual acuity of the patient while the patient completes the visual task.
Clause 143. The system of Clause 142, further comprising a handheld device in electronic communication with the VR headset and the computing device, wherein the handheld device is configured to receive input from the patient as the patient attempts the visual task.
Clause 144. The system of any of Clauses 142 through 143, wherein the one or more eye-tracking sensors and cameras are configured to monitor eye movements of the patient.
Clause 145. The system of any of Clauses 142 through 144, wherein the one or more eye-tracking sensors and cameras are configured to monitor fixation patterns of the patient.
Clause 146. The system of any of Clauses 142 through 145, wherein the one or more eye-tracking cameras comprise infrared cameras.
Clause 147. The system of any of Clauses 142 through 146, wherein the computing device comprises an algorithm that processes the eye data to evaluate a reaction time of the patient.
Clause 148. The system of any of Clauses 142 through 147, wherein the computing device comprises an algorithm that processes the eye data to evaluate an eye movement accuracy of the patient.
Clause 149. The system of any of Clauses 142 through 148, wherein the computing device comprises an algorithm that processes the eye data to evaluate an eye movement stability of the patient.
Clause 150. The system of any of Clauses 142 through 149, wherein the algorithm processes the eye data in real-time.
Clause 151. The system of any of Clauses 142 through 150, wherein the computing device is further configured to generate a report, which is accessible at a user interface of computing device.
Clause 152. A method for evaluating the effectiveness of eye exercises, the method comprising: conducting an initial vision assessment in a virtual environment displayed on screens of a VR headset worn by a patient; monitoring a patient's eyes to collect a patient's responses to the initial vision assessment; conducting one or more eye exercises in the virtual environment; conducting a final vision assessment in the virtual environment; monitoring the patient's eyes to collect a patient's responses to the final vision assessment; and comparing the patient's responses to the initial vision assessment to the patient's responses to the final vision assessment to evaluate changes in a patient's vision.
Clause 153. The method of Clause 152, wherein conducting one or more eye exercises comprises evaluating a length of time for which the patient can focus on an optotype displayed at different depths in the virtual environment.
Clause 154. The method of any of Clauses 152 through 153, wherein conducting one or more eye exercises comprises evaluating whether the patient's eyes are aligned while tracking moving objects.
Clause 155. The method of any of Clauses 152 through 154, wherein conducting one or more eye exercises comprises testing a visual acuity of the patient using an array of optotypes displayed in the virtual environment.
Clause 156. The method of any of Clauses 152 through 155, wherein conducting one or more eye exercises comprises evaluating an extent to which the patient's eyes are coordinated by prompting the patient to fuse objects displayed in the virtual environment.
Clause 157. The method of any of Clauses 152 through 156, wherein monitoring the patient's eyes to collect the patient's responses to the initial and final vision assessments comprises monitoring eye movements of the patient.
Clause 158. The method of any of Clauses 152 through 157, wherein monitoring the patient's eyes to collect the patient's responses to the initial and final vision assessments comprises monitoring focus adjustments of the patient.
Clause 159. The method of any of Clauses 152 through 158, wherein monitoring the patient's eyes to collect the patient's responses to the initial and final vision assessments comprises monitoring a visual acuity of the patient.
Clause 160. The method of any of Clauses 152 through 159, wherein monitoring the patient's eyes to collect the patient's responses to the initial and final vision assessments comprises monitoring the patient's responses in real-time as the patient completes the initial and final vision assessments.
Clause 161. The method of any of Clauses 152 through 160, wherein comparing the patient's responses to the initial vision assessment to the patient's responses to the final vision assessment comprises evaluating eye movements of the patient.
Clause 162. The method of any of Clauses 152 through 161, wherein comparing the patient's responses to the initial vision assessment to the patient's responses to the final vision assessment comprises evaluating focus adjustments of the patient.
Clause 163. The method of any of Clauses 152 through 162, wherein comparing the patient's responses to the initial vision assessment to the patient's responses to the final vision assessment comprises evaluating a visual acuity of the patient.
Clause 164. The method of any of Clauses 152 through 163, further comprising conducting the initial and final vision assessments every time the patient participates in the eye exercises.
Clause 165. The method of any of Clauses 152 through 164, further comprising comparing the patient's responses to the initial and final vision assessments across multiple training sessions.
Clause 166. A system for evaluating the effectiveness of eye exercises, the system comprising: a virtual reality (VR) headset worn by a patient, the VR headset comprising screens, one or more eye-tracking sensors, and one or more eye-tracking cameras, the one or more eye-tracking sensors and cameras being configured to collect eye data; and a computing device in electronic communication with the VR headset, the computing device being configured to: conduct an initial vision assessment, one or more eye exercises, and a final vision assessment on the screens of the VR headset; and process the eye data collected by the one or more eye-tracking sensors and cameras to evaluate an effectiveness of the one or more eye exercises, wherein the eye data comprises eye movements, focus adjustments, and visual acuity of the patient while the patient completes the visual assessment.
Clause 167. The system of Clause 166, further comprising a handheld device in electronic communication with the VR headset and the computing device, wherein the handheld device is configured to receive input from the patient as the patient completes the initial vision assessment, the one or more eye exercises, and the final vision assessment.
Clause 168. The system of any of Clauses 166 through 167, wherein the one or more eye-tracking cameras comprises an infrared camera.
Clause 169. The system of any of Clauses 166 through 168, wherein the one or more eye-tracking cameras comprises at least one eye-tracking camera facing each eye of the patient wearing the VR headset.
Clause 170. The system of any of Clauses 166 through 169, wherein the computing device comprises an algorithm configured to process the eye data to evaluate a visual acuity of the patient.
Clause 171. The system of any of Clauses 166 through 170, wherein the computing device comprises an algorithm configured to process the eye data to evaluate an eye coordination of the patient.
Clause 172. The system of any of Clauses 166 through 171, wherein the computing device comprises an algorithm configured to process the eye data to evaluate a focusing ability of the patient.
Clause 173. The system of any of Clauses 166 through 172, wherein the algorithm is configured to process the eye data in real-time.
Clause 174. A method for identifying and correcting visual stressors in an environment, the method comprising: displaying a virtual environment with a plurality of features on screens of a virtual reality (VR) headset worn by a user; changing a first feature of the plurality of features; upon changing the first feature: displaying a visual task in the virtual environment; prompting the user to complete the visual task; monitoring a user's eyes to collect a first input while the user completes visual task; in response to the input, changing a second feature of the plurality of features; upon changing the second feature: displaying the visual task in the virtual environment again; prompting the user to complete the visual task again; monitoring the user's eyes to collect a second input while the user completes the visual task; wherein features of the plurality of features are changed until the virtual environment is arranged to optimize a visual acuity and an ergonomic comfort of the user.
Clause 175. The method of Clause 174, wherein displaying a virtual environment comprises displaying a VR version of a real environment.
Clause 176. The method of any of Clauses 174 through 175, wherein displaying a virtual environment comprises displaying a virtual office.
Clause 177. The method of any of Clauses 174 through 176, wherein changing the first and second features comprises changing a lighting in the virtual environment.
Clause 178. The method of any of Clauses 174 through 177, wherein changing the first and second features comprises changing a height or size of a desk in the virtual office.
Clause 179. The method of any of Clauses 174 through 178, wherein changing the first and second features comprises changing a height or position of a monitor in the virtual office.
Clause 180. The method of any of Clauses 174 through 179, wherein changing the first and second features comprises changing height or shape of a chair in the virtual office.
Clause 181. The method of any of Clauses 174 through 180, wherein displaying the visual task comprises displaying a task on a virtual computer in the virtual environment.
Clause 182. The method of any of Clauses 174 through 181, wherein displaying the visual task comprises displaying an array of optotypes to evaluate a visual acuity of the user.
Clause 183. The method of any of Clauses 174 through 182, wherein monitoring the user's eyes comprises continuously monitoring the user's eyes.
Clause 184. The method of any of Clauses 174 through 183, wherein monitoring the user's eyes comprises monitoring a gaze direction of the user.
Clause 185. The method of any of Clauses 174 through 184, wherein monitoring the user's eyes comprises monitoring a fixation duration of the user.
Clause 186. The method of any of Clauses 174 through 185, wherein monitoring the user's eyes comprises monitoring a blink rate of the user.
Clause 187. The method of any of Clauses 174 through 186, further comprising processing the first and second inputs to identify sources of visual stress for the user.
Clause 188. The method of any of Clauses 174 through 187, further comprising processing the first and second inputs to identify sources of physical discomfort for the user.
Clause 189. The method of any of Clauses 174 through 188, further comprising changing a type or difficulty of the visual task.
Clause 190. The method of any of Clauses 174 through 189, further comprising asking the user for subjective input regarding the visual acuity and the ergonomic comfort of the user.
Clause 191. The method of any of Clauses 174 through 190, further comprising processing the subjective input to change the features of the virtual environment.
Clause 192. A system for identifying and correcting visual stressors in an environment, the system comprising: a virtual reality (VR) headset worn by a user, the VR headset comprising: screens; one or more motion-tracking sensors configured to track physical movements and a spatial orientation of the user; and one or more eye-tracking sensors and one or more eye-tracking cameras, configured to monitor eye movements, fixation patterns, and blink rates of the user; and a computing device in electronic communication with the VR headset, the computing device being configured to cause a virtual environment with a plurality of features to be displayed on the screens, to cause features of the plurality of features to be changed, and to process the physical movements, the spatial orientation, the eye movements, the fixation patterns, and the blink rates of the user.
Clause 193. The system of Clause 192, further comprising a handheld device in electronic communication with the VR headset and the computing device, wherein the handheld device is configured to receive input from the user as the user completes the visual task.
Clause 194. The system of any of Clauses 192 through 193, wherein the at least one motion-tracking sensor is positioned along a transverse plane of the VR headset, wherein the transverse plane aligns with an eyeline of the user wearing the VR headset.
Clause 195. The system of any of Clauses 192 through 194, wherein the at least one motion-tracking sensor comprises one motion-tracking sensor on a left side of the VR headset, one motion-tracking sensor on a right side of the VR headset, and at least two motion-tracking sensors on a front side of the VR headset.
Clause 196. The system of any of Clauses 192 through 195, wherein the at least one motion-tracking sensor is configured to track a three-dimensional movement of the head of the user.
Clause 197. The system of any of Clauses 192 through 196, wherein the at least one motion-tracking sensor is configured to track physical movements and a spatial orientation of the user in real-time and communicate corresponding data with the computing device in real-time.
Clause 198. The system of any of Clauses 192 through 197, wherein the at least one motion-tracking sensor comprises one or more of an accelerometer or a gyroscope.
Clause 199. The system of any of Clauses 192 through 198, wherein the one or more eye-tracking cameras comprise infrared cameras.
Clause 200. The system of any of Clauses 192 through 199, wherein the one or more eye-tracking cameras comprises at least one eye-tracking camera facing each eye of the user wearing the VR headset.
Clause 201. The system of any of Clauses 192 through 200, wherein the computing device comprises an algorithm configured to process the eye movements, fixation patterns, and blink rates of the user to evaluate visual fatigue of the user.
Clause 202. The system of any of Clauses 192 through 201, wherein the computing device comprises an algorithm configured to process the eye movements, fixation patterns, and blink rates of the user to evaluate eye strain of the user.
Clause 203. The system of any of Clauses 192 through 202, wherein the computing device comprises an algorithm configured to process the physical movements and the spatial orientation to evaluate an ergonomic posture of the user.
Clause 204. The system of any of Clauses 192 through 203, wherein the algorithm is configured to process the eye movements, fixation patterns, blink rates, physical movements, and spatial orientation of the user in real-time.
Clause 205. The system of any of Clauses 192 through 204, wherein the computing device is configured to generate a report summarizing specifications of the virtual environment as optimized for visual acuity and ergonomic comfort.
Clause 206. The system of any of Clauses 192 through 205, wherein the computing device comprises a user interface at which the report can be accessed.
Clause 207. A method for simulating ultraviolet (UV) light exposure, the method comprising displaying a virtual environment on screens of a virtual reality (VR) headset worn by a user and changing lighting conditions of the virtual environment to simulate scenarios in which the user would be exposed to UV light.
Clause 208. The method of Clause 207, wherein changing the lighting conditions to simulate scenarios in which the user would be exposed to UV light comprises increasing a brightness of the virtual environment to simulate sunny outdoor settings.
Clause 209. The method of any of Clauses 207 through 208, wherein changing the lighting conditions to simulate scenarios in which the user would be exposed to UV light comprises displaying a body of water in the virtual environment, wherein light reflects off the body of water and into a user's eyes.
Clause 210. The method of any of Clauses 207 through 209, wherein changing the lighting conditions to simulate scenarios in which the user would be exposed to UV light comprises displaying the virtual environment at a high altitude.
Clause 211. The method of any of Clauses 207 through 210, wherein changing the lighting conditions to simulate scenarios in which the user would be exposed to UV light comprises displaying reflective objects in the virtual environment.
Clause 212. The method of any of Clauses 207 through 211, further comprising teaching the user means for limiting exposure to UV light.
Clause 213. A method for measuring the effects of ultraviolet (UV) light exposure, the method comprising: displaying a virtual environment on screens of a virtual reality (VR) headset worn by a user, wherein the virtual environment comprises lighting conditions that simulate real-life UV light; monitoring a user's gaze direction, blink rate, and pupillary responses with eye-tracking sensors positioned on the VR headset; processing the user's gaze direction, blink rate, and pupillary responses; summarizing the effects of UV light on the user.
Clause 214. The method of Clause 213, wherein monitoring the user's gaze direction, blink rate, and pupillary responses comprises monitoring the user's gaze direction, blink rate, and pupillary responses in real-time as the user reacts to the virtual environment.
Clause 215. The method of any of Clauses 213 through 214, wherein processing the user's gaze direction, blink rate, and pupillary responses comprises evaluating a degree of squinting that the user engages in while reacting to the virtual environment.
Clause 216. The method of any of Clauses 213 through 216, further comprising changing the lighting conditions to simulate different scenarios in which the user might encounter UV light.
Clause 217. The method of any of Clauses 213 through 216, further comprising recommending means for limiting exposure to UV light.
Clause 218. A system for measuring the effects of ultraviolet (UV) light exposure, the system comprising: a virtual reality (VR) headset worn by a user, the VR headset comprising screens, one or more eye-tracking sensors, and one or more eye-tracking cameras, the one or more eye-tracking sensors and cameras being configured to collect eye data; and a computing device in electronic communication with the VR headset, the computing device being configured to cause a virtual environment with lighting conditions that simulate real-life UV light to be displayed on the screens and to process the eye data to adjust the visual task, wherein the eye data comprises gaze direction, blink rate, and pupillary responses of the user.
Clause 219. The system of Clause 218, wherein the one or more eye-tracking cameras comprise infrared cameras.
Clause 220. The system of any of Clauses 218 through 219, wherein the computing device comprises an algorithm configured to process the eye data to evaluate a blink frequency of the user.
Clause 221. The system of any of Clauses 218 through 229, wherein the computing device comprises an algorithm configured to process the eye data to evaluate a degree of squinting of the user.
Clause 222. The system of any of Clauses 218 through 221, wherein the computing device comprises an algorithm configured to process the eye data to evaluate pupil constrictions of the user.
Clause 223. The system of any of Clauses 218 through 222, wherein the algorithm is configured to process the eye data in real-time.
Clause 224. The system of any of Clauses 218 through 223, wherein the computing device configured to generate a report recommending UV protective measures, and the computing device further comprises a user interface at which the report can be accessed.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim can include some or all of the words (e.g., steps, operations, means or components) recited in a clause, a sentence, a phrase or a paragraph. In one aspect, a claim can include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components can include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that can include at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “can include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies, and limits of measurement under the relevant circumstances. In one or more aspects, the terms “about,” “substantially,” and “approximately” may provide an industry-accepted tolerance for their corresponding terms and/or relativity between items.

As used herein, the term “comprising” indicates the presence of the specified integer(s), but allows for the possibility of other integers, unspecified. This term does not imply any particular proportion of the specified integers. Variations of the word “comprising,” such as “comprise” and “can comprise,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) can include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology can include other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method for identifying and correcting visual stressors in an environment, the method comprising:
displaying a virtual environment with a plurality of features on screens of a virtual reality (VR) headset worn by a user;
changing a first feature of the plurality of features;
upon changing the first feature:
displaying a visual task in the virtual environment;
prompting the user to complete the visual task;
monitoring a user's eyes to collect a first input while the user completes visual task;
in response to the input, changing a second feature of the plurality of features;
upon changing the second feature:
displaying the visual task in the virtual environment again;
prompting the user to complete the visual task again;
monitoring the user's eyes to collect a second input while the user completes the visual task;
wherein features of the plurality of features are changed until the virtual environment is arranged to optimize a visual acuity and an ergonomic comfort of the user.

2. The method of claim 1, wherein displaying a virtual environment comprises displaying a virtual office.

3. The method of claim 2, wherein changing the first and second features comprises changing a lighting in the virtual environment.

4. The method of claim 2, wherein changing the first and second features comprises changing a height or size of a desk in the virtual office.

5. The method of claim 2, wherein changing the first and second features comprises changing a height or position of a monitor in the virtual office.

6. The method of claim 2, wherein changing the first and second features comprises changing height or shape of a chair in the virtual office.

7. The method of claim 1, wherein monitoring the user's eyes comprises monitoring a gaze direction of the user.

8. The method of claim 1, wherein monitoring the user's eyes comprises monitoring a fixation duration of the user.

9. The method of claim 1, wherein monitoring the user's eyes comprises monitoring a blink rate of the user.

10. The method of claim 1, further comprising processing the first and second inputs to identify sources of visual stress for the user.

11. The method of claim 1, further comprising processing the first and second inputs to identify sources of physical discomfort for the user.

12. The method of claim 1, further comprising asking the user for subjective input regarding the visual acuity and the ergonomic comfort of the user.

13. The method of claim 12, further comprising processing the subjective input to change the features of the virtual environment.

14. A system for identifying and correcting visual stressors in an environment, the system comprising:
a virtual reality (VR) headset worn by a user, the VR headset comprising:
screens;
one or more motion-tracking sensors configured to track physical movements and a spatial orientation of the user; and
one or more eye-tracking sensors and one or more eye-tracking cameras, configured to monitor eye movements, fixation patterns, and blink rates of the user; and
a computing device in electronic communication with the VR headset, the computing device being configured to cause a virtual environment with a plurality of features to be displayed on the screens, to cause features of the plurality of features to be changed, and to process the physical movements, the spatial orientation, the eye movements, the fixation patterns, and the blink rates of the user.

15. The system of claim 14, wherein the at least one motion-tracking sensor comprises one motion-tracking sensor on a left side of the VR headset, one motion-tracking sensor on a right side of the VR headset, and at least two motion-tracking sensors on a front side of the VR headset.

16. The system of claim 14, wherein the at least one motion-tracking sensor is configured to track physical movements and a spatial orientation of the user in real-time and communicate corresponding data with the computing device in real-time.

17. The system of claim 14, wherein the at least one motion-tracking sensor comprises one or more of an accelerometer or a gyroscope.

18. The system of claim 14, wherein the computing device comprises an algorithm configured to process the eye movements, fixation patterns, and blink rates of the user to evaluate visual fatigue of the user.

19. The system of claim 14, wherein the computing device comprises an algorithm configured to process the eye movements, fixation patterns, and blink rates of the user to evaluate eye strain of the user.

20. The system of claim 14, wherein the computing device comprises an algorithm configured to process the physical movements and the spatial orientation to evaluate an ergonomic posture of the user.
